(19) United States
(12) Patent Application Publication
(10) Pub. No.: US 2011/0287047 A1
(43) Pub. Date: Nov. 24, 2011

(54) CROSS-BETA STRUCTURES AS CARRIER IN VACCINES

(75) Inventors: Barend Bouma, Houten (NL); Johan Renes, Amersfoort (NL); Paulus J.G.M. Steverink, Huizen (NL)

(73) Assignee: Crossbeta Biosciences B.V., CH Utrecht (NL)

(21) Appl. No.: 12/998,691

(22) PCT Filed: Nov. 18, 2009
(86) PCT No.: PCT/NL2009/050696

§ 371 Date: Aug. 5, 2011

(30) Foreign Application Priority Data
Nov. 18, 2008 (EP) 08169365.7

(51) Int. Cl. A61K 39/385; A61P 37/04; C07K 14/00

(52) U.S. Cl. 424/193.1; 530/402

(57) ABSTRACT

The invention provides means and methods for producing and/or selecting immunogenic compositions, comprising providing said composition with at least one epitope for a B-cell receptor and/or at least one epitope for a T-cell receptor, coupled to a protein that comprises at least one crossbeta structure, and testing at least one immunogenic property.

The invention relates to the fields of protein chemistry, immunology and vaccines.

In particular the invention relates to methods for inducing immune responses against desired antigens, pathogens, aberrant cells and the like.

For this purpose, immunogenic complexes, methods for producing them, immunogenic compositions and/or vaccines containing them are provided.

Vaccines are immunogenic compositions the response to which are at least partially effective in preventing infection by a pathogen against which the immune response was elicited, and/or which are capable of at least partially removing from an organism proteins, cells and/or pathogens which are already present in said organism.

Vaccines can be divided in two basic groups, i.e. prophylactic vaccines and therapeutic vaccines. Prophylactic vaccines have been made and/or suggested against essentially every known infectious agent (virus, bacterium, yeast, fungi, parasite, mycoplasm, etc.), which has some pathology in man, pets and/or livestock, which is therefore collectively referred to as pathogen. Therapeutic vaccines have been made and/or suggested for infectious agents as well, but also for treatments of cancer and other aberrancies, as well as for inducing immune responses against other self antigens, as widely ranging as e.g. LHRH for immunocastration of boars, or for use in preventing graft versus host (GvH) and/or transplant rejections.

In vaccines in general there are two vital issues. Vaccines have to be efficacious and vaccines have to be safe. It often seems that the two requirements are mutually exclusive when trying to develop a vaccine. The most efficacious vaccines so far have been modified live infectious agents. These are modified in a manner that their virulence has been reduced (attenuation) to an acceptable level. The vaccine strain of the infectious agent typically does replicate in the host, but at a reduced level, so that the host can mount an adequate immune response, also providing the host with long term immunity against the infectious agent. The downside of attenuated vaccines is that the infectious agents may revert to a more virulent (and thus pathogenic) form.

This may happen in any infectious agent, but is a very serious problem in fast mutating viruses (such as in particular RNA viruses). Another problem with modified live vaccines is that infectious agents often have many different serotypes. It has proven to be difficult in many cases to provide vaccines which elicit an immune response in a host that protects against different serotypes of infectious agents.

Vaccines in which the infectious agent has been killed are often safe, but often their efficacy is mediocre at best, even when the vaccine contains an adjuvant. In general an immune response is enhanced by adding adjuvants (from the Latin adjuvare, meaning “to help”) to the vaccines. The chemical nature of adjuvants, their proposed mode of action and their reactions (side effect) are highly variable. Some of the side effects can be ascribed to an unintentional stimulation of different mechanisms of the immune system whereas others reflect general adverse pharmacological reactions which are more or less expected. There are several types of adjuvants. Today the most common adjuvants for human use are alum (referred to as ‘aluminium hydroxide’ and ‘aluminium phosphate’) and calcium phosphate. However, there is a number of other adjuvants based on oil emulsions, products from bacteria (their synthetic derivatives as well as liposomes) or gram-negative bacteria, endotoxins, cholesterol, fatty acids, aliphatic amines, paraffinic and vegetable oils. Recently, monophosphoryl lipid A, ISCOMs with Quil-A, and Syntex adjuvant formulations (SAFs) containing the threonyl derivative or muramyl dipeptide have been under consideration for use in human vaccines. Chemically, the adjuvants are a highly heterogenous group of compounds with only one thing in common: their ability to enhance the immune response—their adjuvanticity. They are highly variable in terms of how they affect the immune system and how serious their adverse effects are due to the resultant hyperactivation of the immune system. The choice of any of these adjuvants reflects a compromise between a requirement for adjuvanticity and an acceptable low level of adverse reactions. The term adjuvant has been used for any material that can increase the humoral and/or cellular immune response to an antigen. In the conventional vaccines, adjuvants are used to elicit an early, high and long-lasting immune response. The newly developed purified subunit or synthetic vaccines (see below) using biosynthetic, recombinant and other modern technology are poor immunogens and require adjuvants to evoke the immune response. The use of adjuvants enables the use of less antigen to achieve the desired immune response, and this reduces vaccine production costs. With a few exceptions, adjuvants are foreign to the body and cause adverse reactions.

A type of vaccine that has received a lot of attention since the advent of modern biology is the subunit vaccine. In these vaccines only one or a few elements of the infectious agent are used to elicit an immune response. Typically a subunit vaccine comprises one, two or three proteins (glycoproteins) and/or peptides present in proteins or fragments thereof, of an infectious agent (from one or more serotypes) which have been purified from a pathogen or produced by recombinant means and/or synthetic means. Although these vaccines in theory are the most promising safe and efficacious vaccines, in practice efficacy has proved to be a major hurdle.

Molecular biology has provided more alternative methods to arrive at safe and efficacious vaccines that theoretically should also provide cross-protection against different serotypes of infectious agents. Carbohydrate structures derived from infectious agents have been suggested as specific immune response eliciting components of vaccines, as well as lipopolysaccharide structures, and even nucleic acid complexes have been proposed. Although these component vaccines are generally safe, their efficacy and cross-protection over different serotypes has been generally lacking. Combinations of different kinds of components have been suggested (carbohydrates with peptides/proteins and lipopolysaccharide (LPS) with peptides/proteins, optionally with carriers), but so far the safety vs. efficacy issue remains.

Another approach to provide cross protection is to make hybrid infectious agents which comprise antigenic components from two or more serotypes of an infectious agent. These can be and have been produced by modern molecular biology techniques. They can be produced as modified live vaccines, or as vaccines with inactivated or killed pathogens, but also as subunit vaccines. Cocktail or combination vaccines comprising antigens from completely different infectious agents are also well known. In many countries children are routinely vaccinated with cocktail vaccines against e.g. diphteria, whooping cough, tetanus and polio. Recombinant vaccines comprising antigenic elements from different infectious agents have also been suggested. For instance for poultry a vaccine based on a chicken anemia virus has been suggested to be complemented with antigenic elements of Marek disease virus (MDV), but many more combinations have been suggested and produced.

Another important advantage of modern recombinant vaccines is that they have provided the opportunity to produce marker vaccines. Marker vaccines have been provided with an extra element that is not present in wild type infectious agent, or marker vaccines lack an element that is present in wild type infectious agent. The response of a host to both types of marker vaccines can be distinguished (typically by serological diagnosis) from the response against an infection with wild type.

An efficient way of producing immunogenic compositions, or improving the immunogenicity of immunogenic compositions, has been provided in WO 2007/008070. This patent application discloses that the immunogenicity of a composition which comprises amino acid sequences is enhanced by providing said composition with at least one crossbeta structure. A crossbeta structure is a structural element of peptides and proteins, comprising stacked beta sheets, as will be discussed in more detail below. According to WO 2007/008070, the presence of crossbeta structure enhances the immunogenicity of a composition comprising an amino acid sequence. An immunogenic composition is thus prepared by producing a composition which comprises an amino acid sequence, such as a protein containing composition, and administrating (protein comprising) crossbeta structures to said composition. Additionally, or alternatively, crossbeta structure formation in said composition is induced, for instance by changing the pH, salt concentration, reducing agent concentration, temperature, buffer and/or chaotropic agent concentration, and/or combinations of these parameters.

The present invention now provides means and methods to further improve immunogenic compositions by providing a method for producing an immunogenic composition, comprising providing a protein, inducing a crossbeta structure in said protein and providing said protein with at least one exogenous epitope to form an epitope-protein complex and combining said epitope-protein complex with a suitable vehicle for administration to a subject. According to the invention usually two components need to be present in a proteinaceous antigen in order to provide for an improved immune response against such an antigen. On the one hand a crossbeta structure is required to provide for recognition (and probably uptake and direction to processing mechanisms) of the antigen by cells, typically through receptors. On the other hand a recognizable epitope to which the immune response is to be mounted is required (this epitope may be a linear peptide, a conformational (discontinuous) epitope, a hapten, combinations of peptides and/or lipids and/or polysaccharides). Although both epitopes and crossbeta structures may already be present in the proteinaceous antigen by itself (see our earlier applications WO 2007/008070, PCT/NL2008/050709 and PCT/NL2008/050710), the present invention now provides a proteinaceous antigen with exogenous epitopes, also when endogenous epitopes are already present. This enables to better control the presence of epitopes, which may otherwise be lost during induction of crossbeta structures. It also allows for presentation of epitopes from a first antigen to be presented with crossbeta structures and epitopes from a second antigen. This of course may be further developed with epitopes from a third, fourth, etc. antigen.

According to the present invention an immunogenic composition is defined as a composition that elicits an immune response when contacted with components from an immune system, in particular upon administration to a subject. Said immune response may be an innate response, a humoral response, a cellular response or a combination of these. According to the present invention a protein is provided in which crossbeta structures are introduced. Crossbeta structures are defined herein below. A protein according to the invention can be any polypeptide, glycoprotein, complex of subunits, conglomerate of polypeptide chains, etc. It may be based on the full amino acid sequence of a protein or a partial sequence. Crossbeta structures may be induced in any suitable manner, as described herein below. According to the present invention it is preferred that crossbeta structures are induced by means that do not leave traces of inducing substances behind. Such procedures include, but are not limited to changing the pH, salt concentration, reducing agent concentration, temperature, buffer and/or chaotropic agent concentration, and/or combinations of these parameters. A method according to the invention, wherein at least one peptide, peptide-peptide/protein conjugate, lipopeptide, polypeptide, protein, protein-protein conjugate, glycoprotein, carbohydrate-peptide/protein conjugate, peptidoglycan, protein-DNA complex, DNA-peptide/protein conjugate, protein-membrane complex, lipid-peptide/protein conjugate, and/or lipoprotein is subjected to a crossbeta inducing procedure, preferably a change of pH, salt concentration, reducing agent concentration, temperature, buffer and/or chaotropic agent concentration, is therefore also provided. Non-limiting examples of crossbeta inducing procedures are heating, changes in temperature, chemical treatments with e.g. high salt concentrations, exposing to acid or alkaline materials, pressure and other physical treatments. A preferred manner of introducing crossbeta structures in a protein is by one or more treatments, either in combined fashion or sequentially, of heating, freezing, reduction, oxidation, glycation, pegylation, sulphatation, exposure to a chaotropic agent (the chaotropic agent preferably being urea or guanidinium-HCl), phosphorylation, (partial) proteolysis, chemical lysis, preferably with HCl or cyanogenbromide, sonication, dissolving in organic solutions, preferably 1,1,1,3,3,3-hexafluoro-2-propanol and/or trifluoroacetic acid and/or formic acid, either or not followed by a change of solution, or a combination thereof. The protein in which crossbeta structures are induced is provided with at least one exogenous epitope. The exogenous epitope may be derived from the same protein as that in which the crossbeta structure is induced, but it may also be derived from a different protein. It may be a T cell epitope or a B cell epitope. It may also be a sequence of several B and/or T cell epitopes, preferably separated by cleavage sites (string-bead-arrangements). An epitope according to the invention typically comprises less than 100 amino acid residues, whereby the actual epitope is typically less than 50, preferably 25 and for T cell epitopes around 8-13 amino acid residues, typically comprising anchor residues, etc. The actual epitope may be flanked by processing sites, cleavage sites and other sequences necessary and/or beneficial for transport into antigen presenting and/or processing cells.

Exogenous in the context of the epitope means that the epitope is added to the protein in which the crossbeta structures are induced. This addition preferably takes place after induction of said crossbeta structures. In case the epitope is one that is already present in the protein comprising crossbeta structures this means that there will be at least two of these epitopes in the complex, one endogenous and at least one exogenous. The addition may be accomplished in any manner per se. It may be classical chemical coupling by linkers (such as SPDP), it may be on a supporting structure (see below), it may even be recombinantly at the C-terminus or N-terminus of the protein, but this is not preferred. The protein in which the crossbeta structure is induced may be coupled to another protein to form a dimer, or a trimer up to about a pentamer. The other protein may be the same protein, a different protein from the same target, or an indifferent carrier protein. In all cases the coupling will preferably be done before inducing crossbeta structures. An indifferent protein is defined as a protein to which an immune response is not required, but also to which an immune response is essentially not detrimental to the host to which the complex is administered. Such an indifferent protein may be a natural protein (such as ovalbumin, albumin, lysozyme, haemoglobin, (fragments from) fibrin, toxoid) or a synthetic sequence (such as amyloid-beta, like for example amyloid-beta1-22, 1-40, 1-42, 16-22, or amyloid-beta variants with Dutch type mutation E22Q, peptides from fibrin, beta-pep25 (Anginex)). The invention further provides a method according to the invention, wherein the at least one epitope is brought or kept in an immunogenic form by a supporting structure. Epitopes need to be presented to the immune system in a certain conformation. Linear epitopes may adopt this conformation (at least temporarily) spontaneously and therefore may not need a supporting structure. Discontinuous and/or conformational epitopes and/or binding sites are not based on a contiguous sequence of amino acids and therefore their constitutive parts may need to be brought together by a supporting structure. Typically such a structure will comprise several binding sites for C-termini and N-termini of peptides, thereby allowing one or more peptides to be oriented as a loop spanning from one binding site of the supporting structure to another binding site on the supporting structure. For a more detailed description of such supporting structures see patent applications WO 2004/077062(A2), WO 2006/078161(A1) and WO 2008/013454(A2), incorporated herein by reference. However, in principle any supporting structure capable of presenting a conformational and/or discontinuous epitope, that can be linked to the protein in which crossbeta structures are (to be) induced is suitable according to the present invention. Such supporting structures are disclosed in inter alia GB2282813, US2003219451 and US2005159341.

A B-cell epitope or B-cell epitopes and/or a T-cell epitope or T-cell epitopes are also referred to as an “epitope” or “epitopes”.

The invention further provides a method according to the invention, wherein said protein and said at least one epitope are derived from the same antigen, pathogen, and/or aberrant cell. According to the invention an antigen is defined as any proteinaceous structure (inter alia a polypeptide, a glycoprotein, a complex comprising nucleic acid, polypeptides, optionally with lipids and/or polysaccharides, quaternary protein complexes) against which an immune response is desired and/or can be mounted.

In a preferred embodiment the antigen and the exogenous epitope are derived from targets on the same pathological entity, i.a. the same microorganism, the same aberrant cell, or the same antigen. This may enhance the probability of uptake of the target by cells of the immune system and/or of clearance of the complex from the circulation.

It is preferred that the protein in which the crossbeta structure is induced still has relevant epitopes (for recognition and/or clearance of the target) itself, besides the exogenous epitopes that are introduced. Therefore the invention provides a method according to the invention, wherein said crossbeta structure comprising protein comprises relevant endogenous epitopes.

In the alternative, when it is desired that a response is only mounted against the exogenous epitopes, the crossbeta structure containing protein may have no relevant epitopes itself.

The exogenous epitopes may be B cell epitopes, T cell epitopes or, in a preferred embodiment the protein-epitope complex may comprise both T cell and B cell epitopes. Preferably, a T cell epitope is of the right size to be able to fit in the relevant T cell receptor and its MHC partner. If the epitope itself is larger than a peptide that would fit in such a setting, then it needs to be processed by an antigen presenting cell. In that case it is preferred that the epitope comprises the correct processing sites for such an antigen presenting cell. It is furthermore preferred that the T cell epitope comprises the correct anchor residues for fitting in the MHC/T cell receptor.

The same principles of course apply to B cell epitopes.

The invention also provides the results of the methods as described herein. This means that the invention in one of its embodiments provides an epitope-protein complex obtainable by a method as disclosed herein, as well as an immunogenic composition consisting of epitope-protein complexes obtainable by a method disclosed herein and a vehicle suitable for administration. The compositions of the invention do not require adjuvants (although adding substances that give a deposit effect may still be beneficial). It is preferred to add as few components to the composition as possible. Stabilising agents may of course be necessary in aqueous protein solutions. Since the route of administration of compositions according to the invention will often be parenteral and as few components as possible are to be added the preferred vehicle for administration is water for injection.

In a further embodiment the invention provides a method for producing antibodies against at least one desired epitope, comprising preparing an immunogenic composition according to the invention, administering said composition to a nonhuman mammal, isolating B-cells from said nonhuman mammal and generating antibody producing cells and/or antibodies from said B cells in a manner known per se.

Antibodies against at least one desired epitope are for instance made recombinantly or synthetically by applying standard techniques, known to a person skilled in the art, including protein sequence analysis, DNA cloning and expression technology. Standard techniques comprise the following steps: (1) The amino acid sequence, at least from the variable regions of both heavy and light chains, or at least from the complementarity determining regions 1-3 (CDRs), or at least from CDR3 of the heavy chain (HC) of isolated antibodies, is obtained by protein sequence analysis. (2) A nucleic acid sequence, preferably a DNA sequence, encoding the identified amino acids sequence is made synthetically. As an alternative to the exact sequence determined by protein analysis, a sequence can be produced wherein one or more mutations are introduced, preferably in the CDR3, and even more preferably in the CDR3 of the heavy chain (HC), in order to produce antibodies with altered affinity, preferably increased and/or more specific affinity. (3) The nucleic acid is cloned into an appropiate expression vector. Such vector preferably already contains the sequences encoding the constant regions of immunoglobulins of the desired type, such as for instance to obtain IgG1, IgG2a, IgG2b, IgM, IgA, IgE etc. (4) Said vector is transduced in either way into an expression system of choice, preferably a mammalian cell. (5) Cells expressing the antibodies are selected. (6) Recombinantly made antibodies are purified from said cells or cell derived culture supernatant. If mutations are introduced into the original antibody sequence to optimize affinity, the newly made antibodies are optionally re-selected, preferably using a method according to the present invention. Such generation of semi-synthetic antibodies with an even increased repertoire of binding sites, preferably in the complementarity determining regions, preferably in the CDR3, even more preferably in the CDR3 of the HC, is preferably performed by generation of a semi-synthetic library, such as a phage display library (see below).

A combinatorial library can be obtained from any set of antibodies, preferably a set of recombinant antibodies such as those present in a phage display library. Preferably, such a library is comprised of sequences related to mammalian antibodies, preferably human antibodies, like immunoglobulins. In one preferred embodiment, such a phage display library comprising a collection of antibodies is made as follows: firstly, RNA is extracted from B cells or from a tissue comprising B cells. Subsequently, cDNA is prepared. Next, cDNA encoding the variable regions is amplified, cloned into an appropriate phagemid vector and transformed into an appropriate host, such as for example a strain of Escherichia coli. In this way antibodies are expressed, i.e. displayed by phages, as fusion proteins on the surface of filamentous bacteriophages. A phage display library is for instance prepared from B cells obtained from a healthy mammal, preferably a human, mouse, rat or llama, or alternatively from a mammal immunized with an immunogenic composition according to the invention. In this way, a collection of antibodies is prepared with a specific aim to comprise those antibodies specific for infection related or disease related epitopes. For example, in one embodiment a mouse is immunized once or several times with one or a selection of B-cell and/or T-cell epitopes coupled to a crossbeta structure, B cells are isolated from the spleen and used to prepare a phage display library. In another embodiment, B cells are isolated from a human immunized with an immunogenic composition according to the invention. cDNA prepared from these B cells is then preferably used to prepare a phage display library. In such a way a phage display library is prepared to comprise antibodies with specificity for epitopes involved in the chosen infection or disease. For example, a library is prepared with antibodies for the Fc domain of Ig's. In the above described way a person skilled in the art is able to design and prepare a phage display library with any collection of antibodies with emphasis on a particular infection, disease or application.

In one embodiment a phage display library with such a collection of antibodies with an increased repertoire is prepared synthetically. In this way a person skilled in the art is able to design a library comprising antibodies of considerable additional diversity. Preferably, by implementing additional sequences in the hypervariable regions, the CDRs that interact with the antigen epitopes, additional antibodies are made, reshaping the variable domains. Besides antibodies obtained from human sequences, a collection of antibodies is in one embodiment created from any other species, such as llama, camel, alpaca or camelid, to obtain antibodies, such as llama antibodies, also referred to as nanobodies, with properties related to these species. Thus, a phage display library and/or a collection of antibodies is prepared in many ways, for instance from a mammal immunized with one or a set of B-cell and/or T-cell epitopes according to methods of the current invention. In a particularly preferred embodiment, a phage display library and/or a collection of antibodies is prepared from a mammal immunized with an immunogenic composition according to the invention. Antibodies specific for B-cell epitopes and/or T-cell epitopes are preferably selected from a phage display library using means and methods according to the invention, preferably combined with standard procedures for isolating phages. Most straightforward, in a preferred embodiment, epitopes are prepared and are immobilized, and subsequently allowed to bind phages. After extensive washing bound phages are retrieved and amplified by reinfection of host. To allow recovery of only specific phages the selection procedure is preferably repeated several times. Finally, those phages are isolated that are capable of specifically binding B-cell and/or T-cell targets. In a particularly preferred embodiment, antigen comprising epitopes is isolated from a tissue sample obtained from an individual or combination of individuals with a disease or infection. After selection of the appropriate phages DNA encoding the variable regions of the isolated antibodies are preferably isolated from the phagemid DNA in order to generate full antibodies. This is easily performed according to standard procedures. The DNA is preferably cloned into vectors encoding the constant regions for the heavy and light chains. Any vector and any desired type of constant region can be used. The vector is preferably transduced in any known way into an expression system of choice, preferably a mammalian cell. Cells expressing the antibodies are preferably selected. Recombinantly made antibodies are preferably purified from the cells or cell derived culture supernatant. In such a way any immunoglobulin specific for selected epitopes is prepared.

For use in humans, “chimeric” or “humanized” recombinant antibodies are preferably generated. Antibodies obtained from other species are preferably modified in such a way that non-human sequences are replaced with human sequences, wherever possible, while the binding properties of the antibodies are preferably not influenced too much. In one embodiment antibodies are made following immunization strategies according to the invention, preferably using mice or rats, even more preferably using transgenic mice that encode human immunoglobulins. After immunization hybridoma cell lines expressing monoclonal antibodies are preferably prepared by standard procedures, and/or by applying the above described phage display technology. Monoclonal antibodies are preferably selected that are capable of specifically interacting with the B-cell epitopes. “Chimeric” or “humanized” versions of such antibodies, when made using normal mice or rats, are for instance made by replacing the non-human constant regions and the relevant non-human variable regions with the relevant human homologous regions. Moreover, different constant regions are introduced when desired.

Crossbeta structures are present in a subset of misfolded proteins such as for instance amyloid. A misfolded protein is defined herein as a protein with a structure other than a native, non-amyloid, non-crossbeta structure. Hence, a misfolded protein is a protein having a non-native three dimensional structure, and/or a crossbeta structure, and/or an amyloid structure.

Misfolded proteins tend to multimerize. This can result in the formation of amorphous aggregates that can vary greatly in size. In certain cases misfolded proteins are more regular and fibrillar in nature. The term amyloid has initially been introduced to define the fibrils, which are formed from misfolded proteins, and which are found in organs and tissues of patients with the various known misfolding diseases, collectively termed amyloidoses. Commonly, amyloid appears as fibrils with undefined length and with a mean diameter of 10 nm, is deposited extracellularly, stains with the dyes Congo red and Thioflavin T (ThT), shows characteristic green birefringence under polarized light when Congo red is bound, comprises beta-sheet secondary structure, and contains the characteristic crossbeta conformation (see below) as determined by X-ray fiber diffraction analysis. However, since it has been determined that protein misfolding is a more general phenomenon and since many characteristics of misfolded proteins are shared with amyloid, the term amyloid has been used in a broader scope. Now, the term amyloid is also used to define intracellular fibrils and fibrils formed in vitro. Also the terms amyloid-like and amylog are used to indicate misfolded proteins with properties shared with amyloids, but that do not fulfil all criteria for amyloid, as listed above.

In conclusion, misfolded proteins are highly heterogeneous in nature, ranging from monomeric misfolded proteins, to small oligomeric species, sometimes referred to as protofibrils, larger aggregates with amorphous appearance, up to large highly ordered fibrils, all of which appearances can share structural features reminiscent to amyloid.

Amyloid and misfolded proteins that do not fulfil all criteria for being identified as amyloid can share structural and functional features with amyloid and/or with other misfolded proteins. These common features are shared among various misfolded proteins, independent of their varying amino acid sequences and varying amino acid sequence lengths. Shared structural features include for example the binding to certain dyes, such as Congo red, ThT, Thioflavin S, Acridine Orange, Sypro Orange, K114, BTA-1, Chrysamine G, accompanied by enhanced fluorescence of the dyes, multimerization, and the binding to certain proteins, such as tissue-type plasminogen activator (tPA), fibronectin, factor XII, hepatocyte growth factor activator (HGFA), finger domains of tPA, factor XII, fibronectin or HGFA, the receptor for advanced glycation end-products (RAGE), CD36, antibodies and chaperones, such as heat shock proteins, like BiP (grp78 or immunoglobulin heavy chain binding protein). Shared functional activities include the activation of tPA and/or the activation of factor XII and the induction of cellular responses, such as inflammatory responses and an immune response.

A unique hallmark of a subset of misfolded proteins such as for instance amyloid is the presence of the crossbeta conformation or a precursor form of the crossbeta conformation.

A crossbeta structure is a structural element in proteins. A crossbeta structure (also referred to as a “crossbeta conformation”, a “cross-β”, a “cross beta”, “cross-beta” or a “cross-β structure”) is defined as a part of a protein, or a part of an assembly of proteins, which comprises single beta-strands (stage 1) and a (n ordered) group of beta-strands (stage 2), and typically a group of beta-strands, preferably composed of 5-10 beta-strands, arranged in a beta-sheet (stage 3). A crossbeta structure often comprises in particular a group of stacked beta-sheets (stage 4), also referred to as “amyloid”. Typically, in crossbeta structures the stacked beta sheets comprise flat beta sheets in a sense that the screw axis present in beta sheets of native proteins, is partly or completely absent in the beta sheets of stacked beta sheets. A crossbeta structure is formed following formation of a crossbeta structure precursor form upon protein misfolding like for example denaturation, proteolysis or unfolding of proteins. A crossbeta structure precursor is defined as any protein conformation that precedes the formation of any of the aforementioned structural stages of a crossbeta structure. These structural elements present in crossbeta structure (precursor) are typically absent in globular regions of (native parts of) proteins. The presence of crossbeta structure is for example demonstrated with circular dichroism spectropolarimetry (CD), X-ray fibre diffraction or binding of ThT or binding of Congo red, K114, BTA-1, accompanied by enhanced fluorescence of the dyes, or binding of finger domains of tPA, factor XII and fibronectin.

A typical form of a crossbeta structure precursor is a partially or completely misfolded protein. A typical form of a misfolded protein is a partially or completely unfolded protein, a partially refolded protein, a partially or completely aggregated protein, an oligomerized or multimerized protein, or a partially or completely denatured protein. A crossbeta structure or a crossbeta structure precursor can appear as monomeric molecules, dimeric, trimeric, up till oligomeric assemblies of molecules, and can appear as multimeric structures and/or assemblies of molecules.

Crossbeta structure (precursor) in any of the aforementioned states can appear in soluble form in aqueous solutions and/or organic solvents and/or any other solutions. Crossbeta structure (precursor) can also be present as solid state material in solutions, like for example as insoluble aggregates, fibrils, particles, like for example as a suspension or separated in a solid crossbeta structure phase and a soluble phase.

Protein misfolding, formation of crossbeta structure precursor, formation of aggregates or multimers and/or crossbeta structure can occur in any composition comprising peptides with a length of at least 2 amino acid residues, and/or protein(s). The term “peptide” is intended to include oligopeptides as well as polypeptides, and the term “protein” includes proteinaceous molecules including peptides, with and without post-translational modifications such as for instance glycosylation, citrullination, oxidation, lipidation, acetylation and glycation. It also includes lipoproteins and complexes comprising a proteinaceous part, such as for instance protein-nucleic acid complexes (RNA and/or DNA), membrane-protein complexes, etc. As used herein, the term “protein” also encompasses proteinaceous molecules, peptides, oligopeptides and polypeptides. Hence, the use of “protein” or “protein and/or peptide” in this application have the same meaning.

A typical form of stacked beta-sheets is in a fibril-like structure in which the beta-strands are oriented in either the direction of the fiber axis or perpendicular to the direction of the fiber axis. The direction of the stacking of the beta-sheets in crossbeta structures is perpendicular to the long fiber axis.

A crossbeta structure conformation is a signal that triggers a cascade of events that induces clearance and breakdown of the obsolete protein. When clearance is inadequate, unwanted proteins aggregate and form pathologic structures ranging from soluble oligomers up to precipitating fibrils and amorphous plaques. Such crossbeta structure conformation comprising aggregates underlie various diseases and disorders, such as for instance, Huntington's disease, amyloidosis type disease, atherosclerosis, cardiovascular disease, diabetes, bleeding, thrombosis, cancer, sepsis and other inflammatory diseases, rheumatoid arthritis, transmissible spongiform encephalopathies such as Creutzfeldt-Jakob disease, multiple sclerosis, auto-immune diseases, (auto-)immune diseases and/or health problems inflicted by administration of (bio)pharmaceuticles, uveitis, ankylosing spondylitis, diseases associated with loss of memory such as Alzheimer's disease, Parkinson's disease and other neuronal diseases (epilepsy), encephalopathy and systemic amyloidoses.

A crossbeta structure is for instance formed during unfolding and refolding of proteins. Unfolding of proteins occur regularly within an organism. For instance, proteins often unfold and refold spontaneously at the end of their life cycle. Moreover, unfolding and/or refolding is induced by environmental factors such as for instance (a change in) pH, glycation, oxidative stress, salting-in effects, salting-out effects, (change in) protein concentration, citrullination, ischeamia, heat, irradiation, mechanical stress, shear stress, proteolysis, exposure to (foreign) surfaces, a change in contact surface material, and so on. As used herein, the terms crossbeta and crossbeta structure also encompasses any crossbeta structure precursor and any misfolded protein, that possibly comprise a low content of crossbeta structure or does not (yet) comprise crossbeta structure. The term “crossbeta binding molecule” or “molecule capable of specifically binding a crossbeta structure” also encompasses a molecule capable of specifically binding such a misfolded protein or crossbeta structure precursor.

The terms unfolding, refolding and misfolding relate to the three-dimensional structure of a protein. Unfolding means that a protein loses at least part of its three-dimensional structure. The term refolding relates to the coiling back into some kind of three-dimensional structure. By refolding, a protein can regain its native configuration, or an incorrect refolding can occur. The term “incorrect refolding” refers to a situation when a three-dimensional structure other than a native configuration is formed. Incorrect refolding is also called misfolding. Unfolding and refolding of proteins involves the risk of crossbeta structure formation. Formation of crossbeta structures sometimes also occurs directly after protein synthesis, without a correctly folded protein intermediate.

The invention is further explained in the following examples. These examples do not limit the scope of the invention, but merely serve to clarify the invention.

### EXAMPLES

### Abbreviations

ADCC, antibody dependent cell-mediated cytotoxicty; AFM, atomic force microscopy; ANS, 1-anilino-8-naphthalene sulfonate; aPMSF, 4-Amidino-Phenyl)-Methane-Sulfonyl Fluoride; BCA, bicinchoninic acid; bis-ANS, 4,4′-dianilino-1,1′-binaphthyl-5,5′-disulfonic acid; CD, circular dichroism; CE, Crossbeta Epitope; CR, Congo red; CSFV, Classical Swine Fever Virus; DLS, dynamic light scattering; DNA, Deoxyribonucleic acid; dOVA, misfolded ovalbumin comprising crossbeta; ELISA, enzyme linked immuno sorbent assay; ESI-MS, electron spray ionization mass spectrometry; FPLC, fast protein liquid chromatography; FVIII, coagulation factor VIII; g6p, glucose-6-phosphate; GAHAP, alkaline-phosphatase labelled goat anti-human immunoglobulin antibody; h, hour(s); H#, hemagglutinin protein of influenza virus, number #; HBS, HEPES buffered saline; HCV, hepatitis C virus; HGFA, Hepatocyte growth factor activator; HIV, human immunodeficiency virus; HK, Hong kong; HPLC, high performance, or high-pressure liquid chromatography; HPV, human papilloma virus; HRP, horseradish peroxidase; hrs, hours; Ig, immunoglobulin; IgG, immunoglobulin of the class 'G; IgIM, immunoglobulins intramuscular; IgIV, immunoglobulins intravenous; kDa, kilo Dalton; LAL, Limulus Amoebocyte Lysate; MDa, mega Dalton; NMR, nuclear magnetic resonance; ORF, open reading frame; OVA, ovalbumin; PBS, phosphate buffered saline; PCVAD, Porcine Circovirus Associated Diseases; Plg, plasminogen; PRRSV, porcine reproductive and respiratory syndrome virus; RAGE, receptor for advanced glycation end-products; RAMPO, peroxidase labelled rabbit anti-mouse immunoglobulins antibody; RNA, ribonucleic acid; RSV, respiratory syncytial virus; RT, room temperature; SDS-PAGE, sodium-dodecyl sulphate-polyacryl amide gel electrophoresis; SEC, size exclusion chromatography; SWARPO, peroxidase labelled swine anti-rabbit immunoglobulins antibody; TB, tuberculosis; TEM, transmission electron microscopy; ThS, Thioflavin S; ThT, Thioflavin T; tPA, tissue type plasminogen activator; VN, Vietnam; W, tryptophan.

### Detection of Structural Features of Proteins: Crossbeta Detection Assays

### The Presence and Nature of Crossbeta Structure in a Protein is Typically Determined Using One or More of the Following Assays:

### Congo Red Fluorescence

Congo red (CR) is a relatively small molecule (chemical formula: C₃₂H₂₂N₆Na₂O₆S₂) that is commonly used as histological dye for detection of amyloid comprising crossbeta. Congo red is also used to selectively stain protein aggregates with amyloid properties that do not necessarily form fibrils. Congo red is also used in a fluorescence enhancement assay to identify proteins with crossbeta in solution. This assay, also termed Congo red fluorescence measurement, is for example performed as described in patent application WO2007008072, paragraph [101].

### Thioflavin T Fluorescence

Thioflavin T, like Congo red, is used by pathologists to visualize plaques composed of amyloid in tissue sections. It also binds to beta sheets, such as those in amyloid oligomers. The dye is selectively excited at 442 nm, resulting in a fluorescence signal at 482 nm, when bound to crossbeta. It will not undergo this red shift upon binding to precursor monomers or small oligomers, or if there is a high beta sheet content in a non-amyloid context. If no amyloid is present in solution, excitation and emission occur at 342 and 430 nm respectively. Thioflavin T is often used to detect crossbeta in solutions. For example, the Thioflavin T fluorescence enhancement assay, also termed ThT fluorescence measurement, is performed as described in patent application WO2007008072, paragraph [101].

### Thioflavin S Fluorescence

Thioflavin S (ThS), is a dye similar to Thioflavin T and the fluorescence assay is performed essentially similar to ThT and CR fluorescence measurements.

Other Fluorescent Dyes that Bind to Misfolded Proteins

Apart from Congo red, ThT, Thioflavin S, several other dyes bind to misfolded proteins comprising crossbeta structure, resulting in altered fluorescence behavior. Examples are Sypro Orange, Acridine Orange, BTA-1 and K114. Similar to ThT, ThS and Congo red, the dyes Sypro Orange, Acridine Orange, BTA-1 and K114 can be used to sample the presence or occurrence of protein misfolding, i.e. crossbeta, under influence of for example physico-chemical parameters like pH, type of buffer, type and/or concentration of excipients.

### tPA Binding ELISA

tPA binding ELISA with immobilized misfolded proteins; is performed as described in patent application WO2007008070, paragraph [35-36]. One of our first discoveries was that tPA binds specifically to misfolded proteins comprising crossbeta. Binding of tPA to misfolded proteins is mediated by its finger domain. Other finger domains and proteins comprising homologous finger domains are also applicable in a similar ELISA setup (see below).

### BiP Binding ELISA

BiP binding ELISA with immobilized misfolded proteins; is performed as described in patent application WO2007108675, section “Binding of BiP to misfolded proteins with crossbeta structure”, with the modification that BiP purified from cell culture medium using Ni²⁺ based affinity chromatography, is used in the ELISAs. It has been demonstrated previously that chaperones like for example BiP bind specifically to misfolded proteins comprising crossbeta. Other heat shock proteins, such as hsp70, hsp90 are also applicable in a similar ELISA setup.

### IgIV Binding ELISA

Immunoglobulins intravenous (IgIV) binding ELISA with immobilized misfolded proteins; is performed as described in patent application WO2007094668, paragraph [0115-0117]. Alternatively, IgIV that is enriched using an affinity matrix with immobilized protein(s) comprising crossbeta, is used for the binding ELISA with immobilized misfolded proteins (see patent application WO2007094668, paragraph [0143]). It has been demonstrated previously that a subset of immunoglobulins in IgIV binds selectively and specifically to misfolded proteins comprising crossbeta. Other antibodies directed against misfolded proteins are also applicable in a similar ELISA setup.

### Finger Binding ELISA Using Fibronectin Finger Domains

Fibronectin finger 4-5 binding ELISA with immobilized misfolded proteins; is performed as described in patent application WO2007008072. It has been demonstrated previously that finger domains of fibronectin selectively and specifically bind to misfolded proteins comprising crossbeta. In addition to, or alternative to finger domains of fibronectin, finger domains of tPA and/or factor XII and/or hepatocyte growth factor activator are used.

### Factor XII Activation Assay

Factor XII/prekallikrein activation assay is performed as described in patent application WO2007008070, paragraph [31-34]. It has been demonstrated previously that factor XII selectively and specifically bind to misfolded proteins comprising crossbeta, resulting in its activation.

tPA/Plasminogen Activation Assay

Enhancement of tPA/plasminogen activity upon exposure of the two serine proteases to misfolded proteins was determined using a chromogenic assay (see for example patent application WO2006101387, paragraph [0195], patent application WO2007008070, paragraph [31-34], and [Kranenburg et al., 2002, Curr. Biology 12(22), pp. 1833)]. Both tPA and plasminogen act in the Crossbeta Pathway. Enhancement of the activity of the crossbeta binding protease tPA is a measure for the presence of misfolded proteins comprising crossbeta structure. As a control, 4-Amidinophenylmethanesulfonyl fluoride hydrochloride (aPMSF, Sigma, A6664) is added to protein solutions to a final concentration of 1.25 mM from a 5 mM stock. Protein solutions with added aPMSF are kept at 4° C. for 16 h before use in a tPA/plasminogen activation assay. In this way, proteases that are putatively present in protein solutions to be analyzed, and that may act on tPA, plasminogen, plasmin and/or the chromogenic substrate for plasmin, are inactivated, to prevent interference in the assay.

### Binding Assays

Apart from the above described binding assays using crossbeta binding compounds, additional crossbeta binding compounds are used in binding assays for determination of the presence and extent of crossbeta in a sample of a peptide, peptide-peptide/protein conjugate, lipopeptide, polypeptide, protein, protein-protein conjugate, glycoprotein, carbohydrate-peptide/protein conjugate, peptidoglycan, protein-DNA complex, DNA-peptide/protein conjugate, protein-membrane complex, lipid-peptide/protein conjugate and/or lipoprotein. In general, crossbeta binding compounds useful for these determinations are tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo red, K114, CD14, a multiligand receptor such as RAGE or CD36 or CD40 or LOX-1 or TLR2 or TLR4, a crossbeta-specific antibody, preferably crossbeta-specific IgG and/or crossbeta-specific IgM, IgIV, an enriched fraction of IgIV capable of specifically binding a crossbeta structure, Low density lipoprotein Related Protein (LRP), LRP Cluster II, LRP Cluster IV, Scavenger Receptor B-I (SR-BI), SR-A, chrysamine G, a chaperone, a heat shock protein, HSP70, HSP60, HSP90, gp95, calreticulin, a chaperonin, a chaperokine and/or a stress protein. In addition, as disclosed previously in patent application WO2007008072, crossbeta binding compounds for use for the aforementioned determinations are 2-(4′-(methylamino)phenyl)-6-methylbenzothiaziole, styryl dyes, BTA-1, Poly(thiophene acetic acid), conjugated polyeclectrolyte, PTAA-Li, Dehydro-glaucine, Ammophedrine, isoboldine, Thaliporphine, thalicmidine, Haematein, ellagic acid, Ammophedrine HBr, corynanthine, and Orcein.

### Isothermal Titration Calorimetry ((Nano)ITC)

With ITC technology, binding of ligands to molecules in solution is addressed.

Binding constants and number of binding sites for a ligand per molecule are retrieved. The molecule to which ligands bind is soluble or present as insoluble molecules. The buffer is an aqueous solution and can comprise constituents like particulates, lipids, fat, carbohydrates. With ITC for example the availability of epitopes for antibodies on proteins can be scanned, when antibody is titrated to protein in the cell. With ITC for example the presence, number of binding sites and the affinity of fluorescent dyes for proteins comprising crossbeta structure is addressed by titrating dye to the cell with protein. With an ITC apparatus, the interaction and binding of molecules is assessed. Typically, the affinity of a small molecule or protein molecule for a protein in solution, is measured. Experimental settings such as temperature, pH, excipients, protein concentration can be varied.

Measurements of Protein Refolding and/or Changes in Protein Conformation & Multimer Size and Multimer Size Distribution Analysis

### The Size and Nature of a Protein Complex Comprising Crossbeta Structure is Typically Determined Using One or More of the Following Analyses:

### Dual Polarisation Inferometry (DPI)

With DPI, for example multimerization of molecules is monitored in time. Multimerization conditions can be varied. In addition, dimensions of protein molecules or assemblies of protein molecules can be assessed, as well as binding properties to an immobilized ligand. The other way around, also the binding of a protein comprising crossbeta to an immobilized binding partner, e.g. a small molecule ligand or protein ligand, can be assessed.

Quartz Crystal Microbalance with Dissipation Monitoring (QCM-D)

By collecting both the dissipation and the resonance frequency of a quartz crystal,

QCM-D technology is used to characterize the formation of thin films (nm range) such as proteins, onto surfaces, in liquid. This is QCM-D monitoring, performed with sensor technology. In liquid, an adsorbed film may consist of a considerably high amount of water, which is sensed as a mass uptake. Measuring several frequencies and the dissipation reveals whether the adsorbed film is rigid or water-rich (soft). With QCM-D the kinetics of both structural changes and mass changes are obtained, simultaneously.

### Turbidity of Protein Solutions

With turbidity measurements the diffraction of light scattered by protein particles in the sample is detected. Light is scattered by the solid particles and absorbed by dissolved protein. In a turbidity measurement the amount of insoluble particles in a solution is determined. This aspect is used to determine the amount of insoluble protein in samples of protein that is subjected to misfolding conditions, compared to the fraction of insoluble protein in the non-treated reference sample.

### Recording Changes in Binding Characteristics of Binding Partners for a Protein

Antibodies specific for a protein in a certain conformation are used to measure the amount of this protein present in this specific state. Upon treatment of the protein using misfolding conditions, binding of antibodies is inhibited or diminished, which is used as a measure for the progress and extent of misfolding. In addition or alternatively, antibodies are used that are specific for certain conformations and/or post-translational modifications, for example glycation, oxidation, citrullination (gain of binding to the protein subjected to misfolding conditions). When for example glycation and/or oxidation and/or citrullination procedures is/are part of the misfolding procedure, the effect of the treatment with respect to the occurrence of modified amino-acid residues is recorded by determining the relative binding of the antibodies, compared to the non-treated reference protein. Alternatively or in addition to the use of antibodies, any binding partner and/or ligand of the non-treated protein is used similarly, and/or any binding partner and/or ligand other than antibodies, of the misfolded protein is used. When a protein changes conformation ligands or binding partners express altered binding characteristics, which is used as a measure for the extent of protein modification and/or extent of misfolding. This binding of antibodies, ligands and/or binding partners is measured using various techniques, such as direct and/or indirect ELISA, surface plasmon resonance, affinity chromatography, isothermal titration calorimetry, differential scanning calorimetry and immuno-precipitation approaches.

### Differential Scanning Calorimetry/Micro/Nano DSC for Detecting Changes in Protein Conformation

Differential scanning calorimetry (DSC) is a thermo-analytical technique in which the difference in the amount of heat required to increase the temperature of a sample and a reference is measured as a function of temperature. The temperature is linearly increased over time. When the protein in the sample changes its conformation, more or less heat (depending on if it is an endo- or exothermic reaction) will be required to increase the temperature at the same rate as the reference sample. In this way the conformational changes as a result of an increase in temperature can be measured.

### Particle Analyzer

A particle analyzer measures the diffraction of a laser beam when targeted at a sample. The resulting data is transformed by a Fourier transformation and gives information about particle size and shape. When applied to protein solutions, putatively present protein aggregates are detected, when larger than the lower detection limit of the apparatus, for example in the sub-micron range.

### Direct Light Microscope

With a regular direct-light microscope with a preferable magnification range of at least 10×-100×, one can determine visually if there are any protein aggregates present in a sample.

### Photon Correlation Spectroscopy (Dynamic Light Scattering Spectroscopy)

Photon correlation spectroscopy can be used to measure particle size distribution in a sample in the nm-μm range.

### Nuclear Magnetic Resonance Spectroscopy

Nuclear Magnetic Resonance Spectroscopy (NMR) can be used to assess the electromagnetic properties of certain nuclei in proteins. With this technique the resonance frequency and energy absorption of protons in a molecule are measured. From this data structural information about the protein, like angles of certain chemical bonds, the lengths of these bonds and which parts of the protein are internally buried, can be obtained. This information can then be used to calculate the complete three dimensional structure of a protein. This method however is normally restricted to relatively small molecules. However with special techniques like incorporation of specific isotopes and transverse relaxation optimized spectroscopy, much larger proteins can now be studied with NMR.

### X-Ray Diffraction

In X-ray diffraction measurements with protein crystals, the elastic scattering of X-rays from a crystallized protein is measured. In this way in an indirect manner the arrangement of the atoms in the protein can be determined, resulting in a three-dimensional structural model of the protein. First a protein is crystallized and then a diffraction pattern is measured by irradiating the crystallized protein with an X-ray beam. This diffraction pattern is a representation of how the X-ray beam is scattered from the electrons in the crystal. By gradually rotating the crystal in the X-ray beam, the different atomic positions in the crystal can be determined. This results in an electron density map, with which a complete three-dimensional atomic model of the crystallized protein can be calculated, regularly at the 1-3 Å resolution scale. In this model it can be deduced whether protein molecules underwent conformational changes upon treatment with misfolding conditions, when compared to the structural model of the non-treated protein. In addition, modifications of amino-acid residues become apparent in the structural model, as well as whether the protein molecule forms ordered multimers of a defined size, like for example in the range of dimers-octamers.

Determination of the presence of crossbeta in fibers comprising crystallites, and/or in other appearances of protein aggregates comprising at least a fraction of the protein molecules in a crystalline ordering, can be assessed using X-ray fiber diffraction, as for example shown in [Bouma et al., J. Biol. Chem. V278, No. 43, pp. 41810-41819, 2003, “Glycation Induces Formation of Amyloid Crossbeta Structure in Albumin”].

### Fourier Transform Infrared Spectroscopy

Detection of protein secondary structure in Fourier Transform Infrared Spectroscopy (FTIR), an infrared beam is split in two separate beams. One beam is reflected on a fixed mirror, the second on a moving mirror. These two beams together generate an interferogram which consists of every infrared frequency in the spectrum. When transmitted through a sample specific functional groups in the protein adsorb infrared of a specific wavelength. The resulting interferogram must be Fourier transformed, before it can be interpreted. This Fourier transformed interferogram gives a plot of al the different frequencies plotted against their adsorption. This interferogram is specific for the structure of a protein, like a ‘molecular fingerprint’, and provides information on types of atomic bonds present in the molecule, as well as the spatial arrangement of atoms in for example alpha-helices or beta-sheets.

8-Anilino-1-naphthalenesulfonic Acid Fluorescence Enhancement Assay

8-Anilino-1-naphthalenesulfonic acid (ANS) fluorescence enhancement assay, or ANS fluorescence measurement; is performed as described in patent application WO2007094668. Modification: fluorescence is read on a Gemini XPS microplate reader (Molecular Devices).

ANS is a chemical binds to hydrophobic surfaces of a protein and its fluorescence spectrum shifts upon binding. When proteins are in an unfolded state, they generally display more hydrophobic sites, resulting in an increased ANS shift compared to the protein in its native more globular state. ANS can therefore be used to measure protein unfolding.

### Bis-ANS Fluorescence Enhancement Assay

4,4′ dianilio-1,1′ binaphthyl-5,5′ disulfonic acid di-potassium salt (Bis-ANS) fluorescence enhancement assay; is performed as described in patent application WO2007094668. Essentially, bis-ANS has characteristics comparable to ANS, and bis-ANS is also used to probe for differences in solvent exposure of hydrophobic patches of proteins, when measuring bis-ANS binding with a reference protein samples, and with a protein sample subjected to a misfolding procedure.

### Gel Electrophoresis

Gel electrophoresis using sodium dodecyl-sulphate polyacryl amide gels (SDS-PAGE) and Coomassie stain, with various gels with resolutions between for example 100 Da up to several thousands of kDa, provides information on the occurrence of protein modifications and on the occurrence of multimers. Multimers that are not covalently coupled may also appear as monomers upon the assay conditions applied, i.e. heating protein samples in assay buffer comprising SDS. Samples are heated in the presence or absence of a reducing agent like for example dithiothreitol (DTT), when the protein amino-acid sequence comprises cysteines, that can form disulphide bonds upon subjecting the protein to misfolding conditions.

### Western Blot

When antibodies are available that bind to epitopes on the protein under the denaturing conditions as applied during SDS-PAGE, Western blotting is performed with the same protein samples as applied for SDS-PAGE with Coomassie stain, using the same molecular weight cut-off gels, and using the same protein sample handling approaches.

### Centrifugation

Centrifugation and subsequent comparing the protein concentration in the supernatant with respect to the concentration before centrifugation provides insight into the presence of insoluble precipitates in a protein sample. Upon applying increasing g-forces for a constant time, and/or upon applying fixed or increasing g-forces for an increasing time frame, to a protein solution, with analyzing the protein content in between each step, information is gathered about the presence of insoluble multimers. For example, protein solutions are subjected for 10 minutes to 16,000*g, or for 60 minutes to 100,000*g. The first approach is commonly used to prepare protein solutions for, for example use on FPLC columns or in biological assays, with the aim of pelleting insoluble protein aggregates and using the supernatant with soluble protein. It is generally accepted that after applying 100,000*g for 60 minutes to a protein solution, only soluble multimers are left in the supernatant. As multimers ranging from monomers up to huge multimers comprising thousands of protein monomers may all have a density equal to the density of the buffer solution, applying these g-forces to protein solutions does not separate exclusively on size, but on density differences between the solution and the protein multimers.

### Electron Spray Ionization Mass Spectrometry

Electron spray ionization mass spectrometry (ESI-MS) with protein solutions provides information on the multimer size distribution when sizes range from tens of Da up to the MDa range.

### Ultrasonic Spectrometry, or Acoustic Spectrometry

Ultrasonic spectroscopy analysis, for example using an Ichos-II (Process Analysis and Automation, Ltd), provides insight into protein conformation and changes in tertiary structure are measured. In addition the technique can provide information on particle size of protein assemblies, and allows for monitoring protein concentration.

Dialysis (Membranes with Increasing Molecular Weight Cut-Off)

Using one or a series of dialysis membranes with varying molecular weight cut-offs, size distribution/multimer distribution of protein can be assessed at the sub-oligomer scale, depending on the molecular weight of the monomer. Protein concentration analysis between each dialysis step with gradually increasing pore size (suitable for molecular weight ranges between approximately 1000-50000 Da). Protein concentration is for example monitored using BCA or Coomassie+ determinations (Pierce), and/or absorbance measurements at 280 nm, using for example the nanodrop technology (Attana).

Filtration (Filters with Increasing Molecular Weight Cut-Off)

Filtration using a series of filters with gradually increasing MW cut-offs, ranging from the monomer size of the protein under investigation up to the largest MW cut-off available, reveals information on the distribution and presence of protein molecules in multimers in the range from monomers, lower-order multimers and large multimers comprising several hundreds of monomers. For example, filters with a MW cut-off of 1 kDa up to filters with a cut-off of 5 μm (MW's for example 1/3/10/30/50/100/1000 kDa, completed with filters with cut-offs of for example 200/400/1000/5000 nm). In between each subsequent filtration step, protein concentration is assessed using for example the BCA or Coomassie+ method (Pierce), and/or visualization on SDS-PA gel stained with Coomassie, and/or using SEC.

### Transmission Electron Microscopy

Transmission electron microscopy (TEM) is a imaging technique that provides structural information of proteins at a nm to μm scale. With this resolution it is possible to identify the occurrence of protein assemblies ranging from monomers up to multimers of several thousands molecules, depending on the molecular weight of the parent protein molecule. Furthermore, TEM imaging provides insight into the structural appearance of protein multimers. For example, protein multimers appear as rods, globular structures, strings of globular structures, amorphous assemblies, unbranched fibers, commonly termed fibrils, branched fibrils, and/or combinations thereof.

In the current studies, TEM images were collected using a Jeol 1200 EX transmission electron microscope (Jeol Ltd., Tokyo, Japan) at an excitation voltage of 80 kV. For each sample, the formvar and carbon-coated side of a 100-mesh copper or nickel grid was positioned on a 5 μl drop of protein solution for 5 minutes. Afterwards, it was positioned on a 100 μl drop of PBS for 2 minutes, followed by three 2-minute incubations with a 100 μl drop of distilled water. The grids were then stained for 2 minutes with a 100 μl drop of 2% (m/v) methylcellulose with 0.4% uranyl acetate pH 4. Excess fluid was removed by streaking the side of the grids over filter paper, and the grids were subsequently dried under a lamp. Typically, samples are analysed at a magnification of 10K-100K.

### Theoretical Considerations: Estimated Size and Surface of Protein Multimers

The average van der Waals radius of the 20 amino acids is approximately 0.3 nm, or 3 Å. The approximate average volume of an amino acid is 110 Å³. The approximate average surface of an amino acid residue is 28 Å², or 0.28 nm². The approximate average mass of an amino acid residue is 120 Da. From these numbers it is estimated that using the 1.000 kDa MW cut-off filter, at maximum protein assemblies comprising approximately 8500 amino acid residues flow through the filter. This maximum size corresponds to a maximum protein surface on for example a TEM image, of 2400 nm². Assuming a spherical or squaric arrangement of the protein multimer, this corresponds to protein structures with a radius of approximately 27 nm, or 50×50 nm squares, respectively, on TEM images. With for example H5 appearing on the SEC column and on SDS-PA gel as amongst others, 33 kDa and 75 kDa molecules, multimers of up to 30 or 13 H5 monomers will flow through the 1.000 kDa filter, at maximum. By approximation, on average, 1 nm² corresponds to 3.6 amino acid residues or 430 Da, and 1 kDa corresponds to 2.3 nm².

With this approximate numbers it is possible to calculate the number of protein monomers that appear in multimers, as seen for example under the direct light microscope, in SEC fractions, on TEM images and on SDS-PA gels.

In the examples as outlined in this section it is determined whether monomers and/or multimers of the protein comprising crossbeta structure, before or after coupling of epitopes, has dimensions in the range of 0.5 nm to 1000 μm, and more preferably, in the range of 0.5 nm to 100 μm, and even more preferably in the range of 1 nm to 10 μm, and even more preferably in the range of 30-5000 nm. Obviously, this range of dimensions is determined by the number of protein molecules per multimer, with a given number of amino-acid residues per protein molecule. Therefore, the dimensions are alternatively and/or additively expressed in terms of number of protein monomers per multimer. It is clear that these dimensions are suitable for efficient interaction of protein comprising crossbeta structure with APC such as dendritic cells (DCs) (See FIG. 2). Dimensions of assemblies of protein comprising crossbeta structure reminiscent to dimensions of particles that are known to be bound and taken up by APCs as part of an efficient immune response, are preferred. In this context, the sizes of adjuvant particulates, viral particles and bacterial cells, for example, serve as indicative size estimates preferred for protein comprising crossbeta structure.

### Atomic Force Microscopy

Similar to TEM imaging, atomic force microscopy provides insights into the structural appearance of protein molecules at the protein monomer level up to the macroscopic level of large multimers of protein molecules.

### Size Exclusion Chromatography, or Gel Filtration Chromatography

With size exclusion chromatography (SEC) using HPLC and/or FPLC, a qualitative and quantitative insight is obtained about the distribution of protein molecules over monomers up to multimers, with a detectable size limit of the multimers restricted by the type of SEC column that is used. SEC columns are available with the ability to separate molecular sizes in the sub kDa range up to in the MDa range. The type of column is selected based on the molecular weight of the analyzed protein, and on any indicative information at forehand about the expected range of multimeric sizes. Preferably, a reference non-treated protein is compared to a protein that is subjected to misfolding procedures.

### Tryptophan Fluorescence

Assessment of differences in tryptophan (W) fluorescence intensity between two appearances of the same protein provides information on the occurrence of protein folding differences. In general, in globular proteins W residues are mostly buried in the interior of the globular fold. Upon unfolding, refolding, misfolding, W residues tend to become more solvent exposed, which is recorded in the W fluorescence measurement as a change in fluorescent intensity compared to the protein with a more native fold.

### Dynamic Light Scattering

With the Dynamic Light Scattering (DLS) technique, particle size and particle size distribution is assessed. When protein solutions are considered distribution of proteins over a range of multimers ranging from monomers up to multimers is measured, with the upper limit of detected multimer size limited by the resolution of the DLS technique.

### Circular Dichroism Spectropolarimetry

With circular dichroism spectropolarimetry (CD) the relative presence of protein secondary structural elements is determined. Therefore, this technique allows for the comparison of the relative occurrence of alpha-helix, beta-sheet and random coil between a reference protein that is non-treated, and the protein that is subjected to misfolding conditions. An example of a CD experiment for assessment of conformational changes in proteins upon treatment with misfolding conditions is given in [Bouma et al., J. Biol. Chem. V278, No. 43, pp. 41810-41819, 2003, “Glycation Induces Formation of Amyloid Crossbeta Structure in Albumin”]. Devices such as the one developed by Xstalbio (Scotland, UK) are applied for CD measurements with protein samples comprising aggregates, insoluble aggregates, particulates, etc.

### Native Gel Electrophoresis

Distribution over multimers in the range of approximately monomers up to 100-mers is assessed by applying native gel electrophoresis. For this purpose a reference non-treated protein sample is compared to a protein sample which is subjected to a misfolding procedure. When misfolding procedures are applied that introduce modifications on amino-acid residues, like for example but not limited to, glycation or oxidation or citrullination, these changes are becoming apparent on native gels, as well.

Standards for Structure Determinations: Proteins with Crossbeta Structure & Proteins Lacking Crossbeta Structure

For use as positive and negative controls in many of the aforementioned assays, a selection of proteins and peptides is made that either comprise crossbeta structure, or that lack crossbeta structure. For example, the following proteins are implied in assays as controls and references:

1. Amyloid-beta1-42
a. Fibrillar form can be prepared (incubation for hours to days at room temperature, at 1 mg/ml in PBS)
b. Amorphous aggregate can be prepared
c. Synthetic peptide

2. OVA crossbeta form A5
a. OVA is dissolved at 5.2 mg/ml in 20 mM HEPES, 137 mM NaCl, 4 mM KCl. NaOH from a 5 M stock was added to 2% of the total volume. The solution appeared clear. The solution is incubated for 40 minutes at 37° C. (water bath). 5 M HCl stock (2% of the volume) is added to neutralize the added NaOH.
b. not immunogenic in mice (data not shown)

3. dOVA standard
a. OVA is dissolved in PBS to a concentration of 1.0 mg/mL. The solution is kept for 20 min at 37° C. in a water bath and subsequently for 10 min on the roller device (at room temperature). 200 μl aliquots in PCR cups are heat-treated in a PCR machine (MJ Research, PTC-200) (from 30° C. to 85° C. in steps of 5° C. per min). This cycle is repeated 4 times (in total 5 cycles). The samples are subsequently cooled to 4° C. and stored at −80° C.
b. immunogenic in mice (T-cell, titers) (see patent application PCT/NL2008/050710)

4. nOVA standard
a. OVA was dissolved in PBS to a concentration of 1.0 mg/mL. The solution was kept for 20 min at 37° C. in a water bath and subsequently for 10 min on the roller device (at room temperature).
b. low immunogenicity in mice (see patent application PCT/NL2008/050710)

5. FP13
a. Fibrils, 100% beta sheet conformation
b. Synthetic peptide
c. Fibrils obtained upon incubating a 1 mg/ml solution in H₂O or PBS at room temperature or 37° C.
d. See reference 8

6. FP10
a. Random coil, 0% beta sheet
b. Synthetic peptide
c. Solution: 1 mg/ml in PBS or H₂O, kept at 4° C. or at room temperature
d. See reference 8

7. ΔmIAPP
a. 0% beta sheet
b. Synthetic peptide
c. Solution: 1 mg/ml in PBS or H₂O, kept at 4° C. or at room temperature

8. BSA-AGE, Hb-AGE
a. Soluble oligomers with 4-17% beta sheet and crossbeta structure
b. Supernatant after ultracentrifugation comprises multimers
c. Immunogenic in mice (titers) (see for example patent application WO2004004698A2)
d. Glycation of albumin and Hb, to obtain advanced glycation endproducts—protein adducts, is for example performed as follows. For preparation of BSA-AGE, 100 mg ml-¹ of albumin was incubated with phosphate-buffered saline (PBS, 140 mM sodium chloride, 2.7 mM potassium chloride, 10 mM disodium hydrogen phosphate, 1.8 mM potassium di-hydrogen phosphate, pH 7.3) containing 1 M of D-glucose-6-phosphate disodium salt hydrate (anhydrous) (g6p, ICN, Aurora, Ohio, USA) and 0.05% m/v NaN₃, at 37° C. in the dark. The solution is for example glycated for 2-70 weeks. Human Hb at 10 mg/ml is for example incubated for 1-75 weeks at 37° C. with PBS containing 1 M of g6p and 0.05% m/v of NaN₃. After incubations, albumin and Hb solutions were extensively dialysed against distilled water and, subsequently, aliquoted and stored at −20° C. Protein concentrations are for example determined with Advanced protein-assay reagent ADV01 (Cytoskeleton, Denver, Colo., USA).

9. albumin

I. Examples of Proteins that are Provided with Crossbeta Structure, for Subsequent Coupling of Epitopes

The protein comprising crossbeta structure, to which epitopes are coupled, is selected based on several criteria. Access to three-dimensional structure data of the native protein provides the possibility to select proteins that do not comprise beta sheet structure in the native conformation. Applying beta sheet inducing procedures to the protein allows for the detection of beta sheets as a measure of the crossbeta inducing efficiency. Examples of proteins selected based on this criterium are albumin, for example from bovine, human, mouse, rat or rabbit origin, haemoglobin, fibrin FP10 peptide alpha148-157 NH₂-KRLEVDIDIK-COOH [seq. id 1]. Alternatively, from available three-dimensional structure data of native proteins, proteins are selected that comprise beta sheets which are for example positioned in close proximity. Upon applying crossbeta structure inducing methods these beta sheets have a tendency to fold into the crossbeta fold. Another selection criterium is the molecular size of the protein. Small peptides of four amino acid residues can form crossbeta structure in multimeric assemblies, as well as peptides and proteins comprising six amino acid residues per monomer up to several hundreds to thousands amino acid residues per monomer, for example factor VIII, (glycated) albumin, (glycated) haemoglobin, influenza virus haemagglutinin (HA, for example H5), hog cholera virus envelope glycoprotein E2, ovalbumin, immunoglobulins, amyloid-beta, glucagon, a protein antigen of PRRS virus, and any molecular dimension in between. The protein used for forming crossbeta structure has a naturally occurring amino-acid sequence, or has one or more amino-acid mutations. The protein used for crossbeta structure formation is of natural origin, or is obtained using synthetic procedures, or is obtained using recombinant protein production technologies. The protein comprising crossbeta structure has a natural amino-acid sequence, for example without or with one or more mutations, or has a random sequence, for example a scrambled sequence of a naturally occurring protein sequence. When used in an animal, for example in a human, the protein comprising crossbeta structure has a “self” amino-acid sequence, or has a “non-self” sequence, with the sequence originating from a protein present in a different species, or the protein comprising crossbeta structure not occurring in the animal provided with the protein comprising crossbeta.

### Propensity to Form Crossbeta.

Proteins known for their propensity to adopt the crossbeta structure in part of their sequence or in the complete sequence, are selected as part of the invention. Examples are amyloid-beta comprising for example residues 1-40, 1-28, 1-42, 16-22, and for example encompassing mutation of the Dutch type, E22Q, fibrin peptides FP13 alpha148-160 NH₂-KRLEVDIDIKIRS-COOH [seq. id 2], for example with mutation K157V, K157G, K157D, K157A, glucagon, lysozyme and lysozyme point mutants, insulin, islet amyloid polypeptide, endostatin, ovalbumin, influenza virus HA protein, for example H5, factor VIII, platelet factor 4, hog cholera virus envelope glycoprotein E2, albumin, glycated albumin, glycated haemoglobin, immunoglobulins, like for example IgG, immunoglobulin light chains, and any other protein known to a person skilled in the art for being able to adopt the crossbeta structure.

For example, based on the above outlined considerations and selection criteria, the following selection of proteins is used in methods for inducing crossbeta structure and for example used subsequently for coupling to epitopes:

Proteins for Selecting Variants with Varying Crossbeta Structures

1. Albumin
a. Crystal structure available
b. 0% beta sheet in native conformation
c. Crossbeta structures can be induced (e.g. by glycation)

2. OVA
a. Crystal structure available
b. Animal models and cell based assays available
c. Various crossbeta structures can be induced
d. T-cell epitopes known

3. H5
a. Influenza virus challenge models available for, for example mouse and ferret
b. Various crossbeta structures can be formed (see patent applications PCT/NL2008/050709 and PCT/NL2008/050710)
c. T-cell epitopes known
d. Functional antibodies available

4. PRRS virus protein antigen(s)
a. Gp5
b. M
c. Gp5-M heterodimer
d. Gp4
e. Known epitopes
f. Challenge models available

### II. Protocols and Procedures for Misfolding Proteins and Introducing Crossbeta in Proteins

Peptide, peptide-peptide/protein conjugate, lipopeptide, polypeptide, protein, protein-protein conjugate, glycoprotein, carbohydrate-peptide/protein conjugate, peptidoglycan, protein-DNA complex, DNA-peptide/protein conjugate, protein-membrane complex, lipid-peptide/protein conjugate and/or lipoprotein, in summary referred to as ‘protein’ throughout this section, are misfolded with the occurrence of crossbeta structure after subjecting them to various crossbeta-inducing procedures. Below, a summary is given of a non-limiting series of those procedures, which are preferably applied to the proteins used in immunogenic compositions after crossbeta structure is induced.

Misfolding of proteins with the occurrence of crossbeta structure is induced using selected combinations of several parameters. The following parameter settings are for example applied for proteins:
a. protein concentrations ranging from 10 μg/ml to 30 mg/ml, and preferably between 25 μg/ml and 10 mg/ml, and even more preferably between 200 and 2000 microgram/ml
b. pH between 0 and 14, and preferably at pH 1.5-2.5 and/or pH 6.5-7.5 and/or 11.5-12.5 and or at the iso-electric point (IEP) of a protein, and for example established with HCl or NaOH, for example using 2-5 M stock solutions.
c. NaCl concentrations between 0 and 5000 mM, and preferably 125-175 mM
d. buffer selected from PBS, HEPES-buffered saline (20 mM HEPES, 137 mM NaCl, 4 mM KCl, pH 7.4), or no buffer (H₂O),
e. a reducing agent like dithiothreitol (DTT) or 6-mercaptoethanol is incorporated in the reaction mixture, and
f. temperature gradients and temperature end-points for an indicated time frame, that are applied for selected time frames of 10 seconds up to 72 h, and with selected ranges between 0 and 120° C., and preferably between 4 and 95° C., with preferably steps of 0.1-5° C./minute for gradients.

Furthermore, protein misfolding is induced for example by, but not limited to, post-translational modifications like for example glycation, using for example carbohydrates, like for example 50-2000 mM glucose-6-phosphate or glucose or fucose, oxidation, using for example CuSO₄, citrullination, using for example peptidylarginine deiminases, acetylation, sulfatation, (partial) de-sulfatation, (partial) de-glycosylation, enzymatic cleavage, chemical cleavage, polymerization, exposure to chaotropic agents like urea (for example 0.1-8 M) or guanidinium-HCl (for example 0.1-7 M).

Misfolding of proteins with appearance of crossbeta structure is also achieved upon subjecting proteins to exposure to adjuvants currently in use or under investigation for future use in immunogenic compositions. Proteins are exposed to adjuvants only, or the exposure to adjuvants is part of a multi-parameter misfolding procedure accompanied by the formation of crossbeta structure, designed based on the aforementioned parameters and conditions. Non-limiting examples of adjuvants that are implemented in protocols for preparation of immunogenic compositions comprising crossbeta structure are alum (aluminium-hydroxide and/or aluminium-phosphate), MF59, QS21, ISCOM matrix, ISCOM, saponin, QS27, CpG-ODN, flagellin, virus like particles, IMO, ISS, lipopolysaccharides, lipid A and lipid A derivatives, complete Freund's adjuvant, incomplete Freund's adjuvant, calcium-phosphate, Specol.

A typical method for induction of crossbeta structure conformation in a protein is designed as follows in a matrix format, representing a multiparameter sampling space for refining conditions to induce crossbeta structure in a protein, from which preferably subsets of parameter settings are selected.
i. protein concentration is 40/200/1000/5000 μg/ml
ii. pH is 2, 7, 12 and at the IEP of the protein
iii. DTT concentration is 0 or 200 mM
iv. NaCl concentration is 0 or 150 mM
v. urea concentration is 0/2/8 M
vi. buffer is PBS or HBS (with adjusted NaCl concentration and/or pH, when indicated)
vii. temperature gradient is
   a. constantly at 4° C./22° C.-37° C./65° C. for an indicated time
   b. constantly at the melting temperature of the protein, at for example 1-20° C. below or above the melting temperature, for a time frame of for example 10 minutes-72 h
   c. from room temperature to 65° C.-85° C., for 1 to 5 cycles

Subsets of selected parameter settings are for example as follows.
A. 1 mg/ml protein in PBS, pH 7.3, 200 mM DTT, 150 mM NaCl, kept at 37° C. for 60 minutes, followed by dialysis against PBS without DTT
B. 200 μg/ml protein in PBS, 150 mM NaCl, heated in a cyclic manner for three cycli from 25° C. to 85° C., at 0.5° C./minute, with varying pH's.
C. 2 mg/ml protein in H₂O, kept for 1 week at room temperature
   III. Determination and Selection of the Protein Comprising Crossbeta Structure with Defined Percentage Beta Sheet and/or with Defined Size of the Molecular Assembly and/or with Defined Percentage and/or Type of Crossbeta Structure.

The following example outlines the typical steps and procedures for obtaining a protein with crossbeta structure, with defined parameters regarding percentage beta sheet, percentage and type of crossbeta structure, and molecular size of protein assemblies comprising crossbeta structure. In this typical example, bovine serum albumin (‘albumin’) is subjected to crossbeta structure inducing parameters, like for example those depicted in example B. above. An aliquot of the treated albumin is analyzed using circular dichroism spectropolarimetry (‘CD’) and the percentage beta sheet is determined as a measure for the percentage crossbeta structure formed. Another aliquot is subjected to size exclusion chromatography using an Äkta purifier with a Superdex200 column and a S-1000 superfine column (GE Healthcare). Insight is provided regarding the molecular size and the molecular size distribution, and the treated albumin is fractionated based on differences in multimeric size of protein assemblies. The upper molecular weight cut-off of the Superdex200 column is approximately 600 kDa, corresponding to approximately albumin decamers. The range of molecular weights which can be fractionated on the 5-1000 superfine column is approximately 500-100.000 kDa, corresponding to approximately albumin 10-1700-mers. Fractions of treated albumin are analyzed with CD, and those molecular weight assemblies comprising beta sheets are selected. Molecular dimensions are assessed using AFM imaging and/or TEM imaging and/or scanning electron microscopy imaging using for example a Phenom apparatus (FEI Company), and/or direct light microscopy. Exposure of epitopes for antibodies is assessed in an ITC experiment and/or an ELISA and/or a surface Plasmon resonance experiment and/or a DPI experiment. For ITC, the fractions of albumin are brought in the cell and antibody is titrated. In this way it is determined whether the crossbeta inducing procedure left epitopes intact or whether epitopes are shielded. When for example similar procedures as now described for albumin are subjected to H5 protein, functional antibodies capable of neutralizing influenza virus are used in the ELISA and/or the ITC experiment, for scanning of the exposure of functional epitopes in the various treated H5 forms comprising crossbeta structure. The ITC technique and fluorescence measurements are also applied to determine the percentage and type of crossbeta structure present in the treated albumin and fractionated treated albumin, i.e. the crossbeta fingerprint is assessed. The crossbeta fingerprint of a protein comprising crossbeta structure is defined as the binding affinity and number of binding sites per molecule for crossbeta structure binding small molecules, such as for example the dyes CR, ThT, ThS, K115, BTA-1, and compared to controls comprising crossbeta structure and controls without crossbeta structure. For this purpose, in this typical example treated albumin is brought in the cell of the ITC apparatus, and the dyes Congo red, ThT, ThS, Sypro orange, K114, BTA-1, Acridine orange are titrated in separate experiments. The dyes bind to separate unique features of crossbeta structure, and differences in dye binding with respect to number of binding sites per molecule albumin and affinity of the dyes for albumin, amongst various forms of treated albumin show variations in the type and percentage of crossbeta structure. For these assays, standard curves for each dye are established using standard crossbeta structure peptides, like for example amorphously aggregated amyloid-beta1-42, fibrilar amyloid-beta1-42, fibrilar FP13, random coiled FP10, random coiled mouse islet amyloid polypeptide sequence NH₂-SNNLGPVLPP-COOH (ΔmIAPP) [seq. id 3], native albumin. Typically, a peptide comprising 100% crossbeta structure, like for example amyloid-beta1-42 or FP13 and determined with like for example X-ray fiber diffraction, EM imaging, CD, is mixed with a peptide comprising 0% crossbeta structure, like for example FP10 or ΔmIAPP, as determined with for example CD, in a mass/mass ratio ranging from 100-0 to 0-100 with typically steps of 5-25%. Each of the listed dyes is titrated to each ratio of peptides and standard curves of crossbeta structure content against dye binding are constructed. In this way, dye binding to a treated albumin sample is expressed in dye binding units seen with standard crossbeta structure comprising protein. Once the percentage crossbeta structure in albumin molecules in each of the treated albumin fractions is established, a selection can be made of treated albumin forms comprising for example 4 to 75% crossbeta structure in individual protein molecules.

Albumin subjected to crossbeta inducing procedures is analyzed directly according to an example series of structural determinations as outlined above, and/or after inducing crossbeta structure, treated albumin is analyzed after being it subjected to gravitational forces, like for example for 10 minutes at 10,000-18,000*g, or for 1 h at 50,000-250,000*g, and/or after filtration using molecular weight cut-off filters in for example the range 100-1.000 kDa. For example, applying g-forces and filtering the treated albumin are conducted one after another in any of the two possible orders, before samples are subjected to structural analyses and/or SEC fractionation followed by subsequent structural analyses. After centrifugation steps, both the soluble fraction and the resuspended pellet fraction are subjected to molecular size and structure analyses. After filtration steps, both the filter flow-through and the filter retentate are subjected to molecular size and structure analyses.

Basically similar to the approach outlined above for albumin, protein antigens of PRRS virus, for example glycoprotein gp4, gp5, matrix protein M and/or the gp5-M heterodimer and/or the other PRRSV proteins outlined in Table 1 and 2, are subjected to crossbeta structure inducing procedures and the type and amount of crossbeta structure is determined, as well as the multimeric size distribution and the molecular appearance. Selected forms of PRRS virus protein antigens comprising crossbeta structure are introduced in the antigen presenting cell (APC)-based screening assay for determination of the molecular size of proteins comprising crossbeta structure, required for efficient immune potentiation.

In addition to formation of albumin and PRRSV protein antigens comprising various forms of crossbeta structure, also H5 protein of various H5N1 strains is subjected to crossbeta inducing procedures. Selected H5 is for example present in viral strains A/HK/156/97 or A/VN/1203/04.

IV. Determining Dimensions of Proteins Comprising Crossbeta Structure (molecular/particle size, form) suitable for binding, Internalization/Engulfment by Antigen Presenting Cells, Like Dendritic Cells, or by Accessory Innate Immune Cells.

### Cell-Based Assay for Evaluation of Immune-Potentiating Properties of Proteins Comprising Crossbeta Structure

In order to select efficient immune system activating and efficient immune potentiating proteins comprising crossbeta structure, selected proteins comprising crossbeta structure are tested in an in vitro cell culture system. The various proteins comprising varying crossbeta structure are evaluated for their capacity to activate antigen presenting cells and to be internalized by antigen-presenting cells (APCs), including for example dendritic cells and monocyte/macrophage type cells. The later cells contribute for example directly or indirectly to antigen-presentation executed by dendritic cells. They act amongst other activities as accessory cells, which, up-on binding and internalization of protein comprising crossbeta structure, become activated to release soluble or contact factors that eventually stimulate receptors carried by the antigen-presenting dendritic cells. In principal, other innate immune cells, including neutrophils, eosinophils, mastcells, NK cells, NKT cells, etc., become activated by the protein comprising crossbeta structure as well and contribute indirectly to antigen presentation executed by dendritic cells. Effective immune potentiation of APCs by protein comprising crossbeta structure is also assessed in in vivo experiments, as outlined below.

### Experimental Approach

Dendritic cells of human or murine origin, or derived from a species of veterinary relevance, are obtained by standard procedures, from either peripheral blood mononuclear cells (PBMC), bone marrow, or other lymphoid sources. They are cultured in the presence of the protein comprising crossbeta structure and monitored for internalization of the protein comprising crossbeta structure. Internalization is monitored by tracking of labeled protein comprising crossbeta structure, after several time points, preferably 6, and 24 hours after exposure. For example, presence of protein comprising crossbeta structure in and/or at cell surfaces is assessed by applying flow cytometry using a FACScalibur (BD Bioscience).

In addition, we monitor dendritic cell activation, using well-described markers of activation. These include up-regulation of cell surface marker expression such as CD80, CD83, CD86, CD40 etc., as well as secreted soluble factors such as cytokines, including TNF-alpha, IL-1, IL-6, IL-18, IL-12, chemokines, or nitrate (NO) or oxygen radicals.

### Details:

### Generation of DCs

For example, murine dendritic cells are cultured from bone marrow according to established methods. Briefly, bone marrow cells are isolated from either Balb/C of C57BL/6 murine femurs, and cultured at 1×10⁶ cells per ml RPMI 1640 medium containing 10% FBS 501 U/ml pencillin (RPMI⁺) in the presence of 10 ng/ml GM-CSF (PMC2016, Bioscource). At day 7 DCs (DC7) differentiation and maturation state is confirmed by cell surface expression of CD11c⁺/CD11b⁺ and CD86^{lo}/CD32/16^{hi} and MHCII^{lo} expression respectively. Therefore, DC are stained with a panel of fluorochrome-conjugated Abs as indicated, all purchased at PharMingen (PharMingen SanDiego, Calif.). Non-specific FcR binding is prevented with FcR blocking Ab, clone 2.4G2 (Pharmingen).

### V. Selection of Epitopes

Resources containing sequence information on B-cell epitopes and T-cell epitopes are known to a person skilled in the art, and a non-limiting summary of examples of epitopes are listed in Table 1 and 2. For example, T-cell epitopes can be predicted using prediction software known to a person skilled in the art. For example, tumor-specific or tumor related epitopes are known to a person skilled in the art. The degree of detail of the knowledge on the exact epitope sequence varies from pathogen to pathogen and from protein antigen to protein antigen: for some pathogens or aberrancies, only the protein antigen that comprises epitopes is known, whereas for other pathogens and protein antigens amino-acid sequences spanning B-cell epitopes and/or spanning T-cell epitopes are known.

Any of the antigen proteins comprising the epitopes, and listed below and/or in the Tables, are not only candidates for selection of B-cell epitopes and/or T-cell epitopes, but are also candidates for selection as the protein comprising crossbeta structure to which exogenous epitopes are coupled. For this purpose, the antigen protein or protein fragment is subjected to crossbeta structure inducing procedures, as outlined above, and subsequently, epitopes are coupled.

Epitopes are selected from for example antigens of pathogens or for example from disease-related proteins or health problem related proteins, like for example epitopes related to those antigens, diseases, pathogens, health problems listed here:
HIV (epitopes from for example gp120)
malaria
pediatric diseases
measles
mumps
Lyme
Hepatitis B
Hepatitis C
herpes simplex type 2
influenza
MRSA
(ox)LDL
West Nile virus
pneumococcus (streptococcus)
TBC/mycobacterium
cytomegalo virus
RSV
meningococcus/Neisseria meningitidis
blue tongue
foot & mouth disease
PRRS virus
rotavirus/traveller diarrhoea
feline infectious peritonitis virus (FIPV)
classical swine fever virus
fasciola hepatica infection
smoking/nicotine
alcohol addiction
acne
allergens
haptens
amyloid proteins, e.g. oxLDL, involved in atherosclerosis
angiotensin
fat molecules/cholesterol, related to (the risk for) health problems
tumor antigens
prostate antigens
HPV
H#N#, various clades, for example H5N1 of Glade 1, 2, 3, and/or with H# being for example H1, H2, H3, H5, H7, H9 and/or with N# being for example N1, N2, N7, N9

### Cervix Cancer

For immunogenic compositions, for coupling to protein comprising crossbeta structure, epitopes of Human papilloma virus (HPV), related to cervix cancer, are for example selected from:

HPV-16 L1 protein
HPV-18 L1 protein
HPV Type 6 L1 protein
HPV-11 L1 protein
HPV E6 antigen
HPV E7 antigen

Epitopes of HPV are also depicted in patent applications U.S. Pat. No. 7,153,659 and WO2004/105681.

### Influenza

Immunogenic compositions comprising epitopes of influenza virus comprise linear and/or non-linear epitopes for B-cell receptors, and/or comprise epitopes for receptors of CD4+ T-cells, and/or comprise epitopes for receptors of CD8+ T-cells, for which the epitopes originate from antigen protein HA, and/or NA, and/or M1, and/or NP, and/or PA, and/or M2, and/or NS1, and/or NS2, and/or PB1, and/or PB2. Antibody epitopes are identified from for example the virus surface proteins HA, NA and M2. Host species for which influenza virus epitopes are selected are for example human, ferret, mouse, monkey, rabbit, chicken, goat. Influenza virus strains from which epitopes are selected are for example influenza B virus type of epitopes or influenza A virus type of epitopes, for example epitopes originating from influenza A virus strains H1N1, H1N9, H2N2, H3N2, H3N8, H5N1, H5N2, H5N9, H7N1, H7N7, H9N2, H11N9 or H13N9. Epitopes of influenza virus can originate from any virus isolate available, like for example but not limited to H5N1 strains A/HK/156/97 and A/VN/1194/04. For example, two epitopes are outlined in detail:

Epitope IYSTVASSL (epitope present in various H5N1 strains, for example in H5 of H5N1 strain A/VN/1203/04)
Epitope LGVSSACPYQGKSSF (epitope from H5 of H5N1 strain A/VN/1203/04)

See reference 1. and 2. for a review of linear and conformational antibody epitopes, and CD4+ or CD8+ T-cell epitopes of influenza virus. In this review information is provided, if available at the time (May 22, 2006), on whether the antibody epitopes or T-cell epitopes are protective epitopes. For the T-cell epitopes the MHC restriction alleles are also provided.

### Prostate Cancer

For other immunogenic compositions, for coupling to protein comprising crossbeta structure, epitopes are selected from Prostate cancer antigens like for example:
prostate specific antigen, PSA, also referred to as kallikrein 3 (KLK3)
Thomsen-Friedenreich (TF) antigen
carbohydrate
prostate stem cell antigen
prostatic acid phosphatase (PAP) CTL epitope
six-transmembrane epithelial antigen of prostate (STEAP) CTL epitope
MUC-1-32mer (-CHGVTSAPDTRPAPGSTAPPAHGVTSAPDTRPA), 8-fold glycosylated with Tn using the -T2 and -T4_-N-acetylgalactosaminyltransferases
Globo H
Lewis^{y}
Tn(c)
TF(c) clusters
GM2 (rabbit brain)
antibodies against T-cell inhibitory receptor CTLA-4
prostate-specific membrane antigen (PSMA)
glycoprotein antigens such as the mucins (MUC-1 and -2), PSA, prostate-specific membrane antigen, acid phosphatase, prostate stem cell antigen
glycolipids such as globo H, GM-2, Lewisy, and Tn, and Thomsen-Friedenreich antigens
kallikrein 4
prostein

For example for PRRS virus, the proteins GP3, GP4, GP5, M, N, Nsp1, Nsp2 or Nsp7 or the gp5-M heterodimer are suitable as proteins comprising crossbeta structure in immunogenic compositions, when crossbeta structure is induced in the proteins and epitopes are coupled. Epitopes selected from gp4, gp5 and/or M are for example coupled to the PRRS virus protein antigen comprising crossbeta structure, and/or PRRS virus epitopes are coupled to protein comprising crossbeta structure that is not a protein from the same pathogen, like for example albumin, ovalbumin, FP13.

Based on the aforementioned listing of the non-limiting amount of data available on known B-cell epitopes and known T-cell epitopes, for example the following antigens are selected as a source of B-cell and/or T-cell epitopes:

### Example

### Selected Antigens as a Source of Indicated Epitopes for Coupling to Proteins Comprising Crossbeta Structure

1. influenza virus antigens
a. example: epitope NH₂-IYSTVASSL-COOH
b. example: epitope NH₂-TYISVGTST-COOH
c. example: epitope NH₂-KYVKSNRLV-COOH
d. example: epitope NH₂-DYEELKHLL-COOH
e. example: epitope NH₂-SYNNTNQEDL-COOH
f. example: epitope NH₂-TYISVGTSTL-COOH
g. example: epitope NH₂-KYVKSNRLVL-COOH

2. OVA
a. Example: T-cell epitope NH₂-VAAHAEINEA-COOH
b. Example: T-cell epitope NH₂-SIINFEKL-COOH
c. Example: T-cell epitope NH₂-AAHAEINEAG-COOH

3. PRRS virus antigens
a. Example: see indicated epitopes in Table 1
b. Example: see indicated epitopes in Table 2
   VI. Methods for Preparing Protein with Crossbeta Structure, with Coupled Epitopes

Below, non-limiting examples are provided of selected combinations of a protein provided with crossbeta structure and with coupled one or more epitopes:
A. OVA provided with crossbeta structure with coupled relevant peptide epitopes
   1. HPV B-cell epitope(s)
   2. HPV T-cell epitope(s) (known epitopes and/or a library of overlapping sequences)
   3. HPV B-cell and T-cell epitope(s)
B. PRRS virus antigen protein comprising crossbeta structure provided with coupled PRRS virus epitope(s)
   1. B-cell epitope(s)
   2. T-cell epitope(s)
   3. Combined B-cell epitope(s) and T-cell epitope(s)
C. Crossbeta protein with coupled H5 protein comprising epitopes and/or with coupled haemagglutinin protein of any influenza A or B strain (with known B-cell epitope(s) and/or T-cell epitope(s)), and/or with coupled B-cell epitope(s) and/or T-cell epitope(s) selected from references 1-4 or Table 1 and 2.
D. Albumin with crossbeta structure with coupled epitopes
E. Amyloid-beta comprising crossbeta structure with coupled one or more epitopes like for example a coupled amyloid-beta B-cell epitope

In FIGS. 1 and 3, examples are provided for various forms of protein comprising crossbeta structure with one or more coupled epitopes. Expanding on these examples, for example, proteins used as the carrier protein comprising crossbeta structure, and therefore subjected to crossbeta inducing procedures, are selected from the listing albumin, ovalbumin, βpep25 (Anginex), FP13, fibrin peptide FP6 with sequence NH₂-IDIKIR-COOH [seq. id 4] Aβ1-40, Aβ1-42, haemoglobin, H5, H1, H3, insulin, lysozyme, glucagon. Moreover, any antigen protein of a pathogen from for example those listed above, is selected as the protein in which crossbeta structure is induced and to which epitopes are coupled.

### Coupling of an Epitope or Epitopes to Protein Comprising Crossbeta Structure: Methods and Techniques

Below, a non-limiting summary is outlined of several coupling techniques available and known to a person skilled in the art for coupling of epitopes to a suitable protein carrier, i.e. according to the current invention a protein comprising crossbeta structure.

Chemically Linked Peptides on Scaffolds (CLIPS) for Reconstruction of Complex Interaction Sites on Molecular Targets Comprising Linear Epitopes and/or Conformational Epitopes and/or Discontinuous Epitopes
CLIPS is a coupling method developed by Pepscan, the Netherlands, in which one or more synthetic peptides are coupled to a chemical scaffold in such a way they mimic complex epitopes. In this method, peptides containing one or more cysteine groups, are coupled to bis-, tris-, and tetrakis(bromomethyl)-benzene derivatives. The cysteine groups will couple to the bromo groups, resulting in specific conformations of the peptides. See also references 5, 6.

Click Chemistry

Click chemistry was developed in 2001 and essentially is a new way of looking at chemical synthesis. It is based on the use of chemical building blocks with high energy content, which can spontaneously form covalent bonds together. Proteins can also be modified to contain ‘Click reactive’ groups. For instance, one of the most classical Click Chemistry reactions is the cycloaddition of alkynes and azides to yield 1,2,3-triazoles. These azide and alkyne groups are quite easily introduced in organic compounds, which makes Click Chemistry a useful method for coupling proteins together. See also: pubs.acs.org/cen/coverstory/8006/8006clickchemistry.html and www.rsc.org/publishing/journals/CS/article.asp?doi=b613014n

Native Chemical Ligation; Coupling Peptides Through an α-Thioester and a Cysteine Residue

Native Chemical Ligation is a widely used technique for coupling polypeptides. It is based on the reaction of a thioester on one peptide with a cysteine residue on the other peptide. Under influence of thiol catalyst, after the formation of a thioester-linked intermediate, an amide bond is formed between the two residues. See also: http://en.wikipedia.org/wiki/Native_chemical_ligation.

Glutaraldehyde Coupling

Glutaraldehyde is a chemical compound which has an amino binding group at each side of the molecule, which can bind peptides and proteins. The reaction is non-catalyzed and can be stopped by adding a primary amine solution (such as ethanolamine). Amine groups bind reactive aldehyde groups that are still present. Further information can be retrieved from www.piercenet.com

Glutaraldehyde/NaBH₄ Coupling

Sometimes a reducing chemical agent such as NaBH4 is added which reduces the aldehyde groups. This also stops the reaction. Another benefit from this reaction is that by reducing the active aldehyde groups, the fluorescence of the glutaraldehyde is also reduced.

100 mM N-hydroxysuccinimide (NHS) and 400 mM N-ethyl-N′-(dimethyl-aminopropyl)-carbodiimide (EDC)

EDC is a chemical which reacts with carboxyl groups to form an amine-reactive O-acylisourea group. This can be used to couple carboxyl groups to amine groups. The O-acylisourea intermediate is however also susceptible to hydrolysis, and thus very unstable in aqueous solutions. Therefore NHS is added, resulting in the formation of a semi-stable amine-reactive sulfo-NHS ester from the O-acylisourea intermediate, thus increasing the coupling efficiency. See also www.piercenet.com/Objects/View.cfm?type=ProductFamily&ID=02030312.

Introduction or Use of an Amino-Terminal Cysteine for Coupling Purposes to Maleimide Activated Carrier Protein

Maleimide is a chemical compound which binds sulfhydryl groups under neutral pH. Cysteine residues contain such groups. Maleimide activated carrier protein thus can be used to bind other proteins. When the reaction pH is higher than 8.5, maleimide preferably reacts to primary amine groups instead of sulfhydryl groups. A carrier protein thus can be activated by maleimide at high pH (maleimede couples to amine groups) and then be used at neutral pH to couple to cysteines of another protein. Sulfyhdryl groups can also be chemically induced. See also www.piercenet.com/Proteomics/browse.cfm?fldID=CE4D 6C5C-5946-4814-9904-C46E01232683 and/or www.piercenet.com/files/ELISAHB1601158pt3.pdf.

Cyanogen Bromide (CNBr)

Cyanogen bromide reacts with hydroxyl groups on for example carriers such as Sepharose, and form cyanate esters or imidiocarbonates. These cyanate esters and imidiocarbonates in turn will readily react with primary amine groups on proteins, resulting in covalent coupling. See for example www.sigmaaldrich.com/sigma/product%20information%20sheet/c9210pis.pdf

Aldehyde

An aldehyde is an organic compound containing a terminal carbonyl group. These aldehyde groups can form covalent bonds with side chain amino groups on peptides and proteins. Aldehyde groups can for instance be made by oxidating sugars with periodate. This makes this coupling method especially useful for coupling of proteins to sugar groups or glycoproteins. See www.pubmedcentral.nih.gov/pagerender.fcgi?artid=1455952&pageindex=1.

Epoxy

Epoxy is a polymer consisting of two carbon atoms and one oxygen atom in a ring like structure. The advantage of epoxy groups for the use in coupling is, that they can covalently bind amino-, thiol- and hydroxyl groups, making it a very versatile coupling agent. See for example stanxterm.aecom.yu.edu/wiki/index.php?page=Coupling_of_proteins_or_peptides and/or wolfson.huji.ac.il/purification/PDF/affinity/SARTORIUS_SartobindEpoxy75.pdf.

Azlactone

Azlactones are cyclic N-acyl-α-amino acids and react spontaneously with primary amine groups on proteins and peptides to form very stable covalent amide bonds. See also: www.piercenet.com/products/browse.cfm?fldID=1846204A-8A84-4AD2-A479-BB3808886BDE.

Biotin/Streptavidin

Biotin is a water soluble vitamin which binds with high affinity to streptavidin, a protein from Streptomyces avidinii. These agents can be used for coupling proteins, by biotinylation of different proteins and/or peptides and using streptavidin as a coupling agent. Streptavidin can bind up to four biotin molecules. Biotinylaton of proteins and peptides is done by making an NHS- or sulfo-NHS-ester on a side chain of biotin, which in turn can bind amine groups on the protein or peptides. Various alternative methods are also in use. See for further information www.piercenet.com/Objects/view.cfm?type=Page&ID=83EFA139-8363-40F8-9F 7D-A689125C9EBA and/or www.piercenet.com/Proteomics/browse.cfm?fldID=84EBE112-F871-4CA5-807F-47327153CFCB.

### Antibodies Suitable for Probing the Exposure of B-Cell Epitopes Coupled to Protein Comprising Crossbeta Structure

After providing a protein comprising crossbeta structure with epitopes through coupling using for example any of the methods outlined above, it is preferably tested whether coupled B-cell epitopes are still accessible for antibodies after coupling. This is assessed using available (functional) antibodies specific for the coupled epitopes. For this purpose, the protein comprising crossbeta structure with coupled epitopes is analyzed for binding of antibodies using for example an ELISA lay-out and/or for example an ITC experiment, in which antibody is titrated to either the free epitope, or to the complex of protein comprising crossbeta structure and epitopes. For further use, those proteins comprising crossbeta structure with coupled epitopes that have epitopes freely accessible are selected for immune assays (see below).

PRRS virus protein antigens comprising crossbeta structure and with coupled epitopes: For example, for selecting immunogenic compositions for providing protection against PRRSV infection, PRRSV protein antigen comprising crossbeta structure and with coupled B-cell epitopes is subjected to a binding study using functional antibodies, which means that antibodies against the coupled epitopes are used, which neutralize the PRRSV. Those immunogenic compositions are selected that comprise B-cell epitopes that are readily accessible for binding of the functional antibodies.

For example, for selection of immunogenic compositions having a greater chance of being capable of eliciting a protective prophylactic immune response against infection with CSFV, for example strain Brescia 456610, in animals, for example in mice and/or in pigs, the following mouse monoclonal antibodies are implicated in the screenings.

### CediCon CSFV 21.2,

### CediCon CSFV 39.5 and

### Cedicon CSFV 44.3,

purchased from Prionics-Lelystad, and which neutralize CSFV in vitro (information from the manufacturer).

For example, for selection of immunogenic compositions having a greater chance of being capable of eliciting an immune response against a protein, for example OVA, in animals, for example in mice and/or in rabbits, the following mouse monoclonal antibodies and polyclonal antibodies are implicated in the screenings for those immunogenic compositions that comprise exposed functional epitopes.
mouse HYB 099-01 (IgG1) The epitope specificity differs from that of HYB 099-02 and HYB 099-09.
mouse HYB 099-02 (IgG1) The epitope specificity differs from that of HYB 099-01 and HYB 099-09.
mouse HYB 099-09 (IgG1)
goat IgG fraction 55303, 5 mg/ml (MP Biomedicals)
rabbit IgG fraction 55304, 4 mg/ml (MP Biomedicals)

For example, for selection of immunogenic compositions having a greater chance of being capable of eliciting a protective prophylactic immune response against infection with influenza virus H5N1 strain A/VN/1203/04 or strain A/HK/156/97 in mice and/or in ferrets, the following mouse monoclonal antibodies, that are affinity purified, are implicated in the screenings.

Rockland anti-H5 A/VN/1203/04 catalogue number 200-301-975, 1 mg/ml (Tebu-bio 12467)
Rockland anti-H5 A/VN/1203/04 catalogue number 200-301-976, 1 mg/ml (Tebu-bio 12468)
Rockland anti-H5 A/VN/1203/04 catalogue number 200-301-977, 1 mg/ml (Tebu-bio 12469)
Rockland anti-H5 A/VN/1203/04 catalogue number 200-301-978, 1 mg/ml (Tebu-bio 12470)
Rockland anti-H5 A/VN/1203/04 catalogue number 200-301-979, 1 mg/ml (Tebu-Bio 12471)
HyTest IgG2a clone 8D2
HyTest clone 17C8
HyTest IgG2a clone 15A6

The anti-H5 antibodies purchased from Rockland inhibit hemagglutination and neutralize H5N1 A/VN/1203/04 virus, according to the supplied datasheets. Antibodies purchased from HyTest inhibit hemagglutination when H5N1 of the strains A/VN/1203/04 or A/HK/156/97 is used, according to information from the manufacturer.

VII. Protein Comprising Crossbeta Structure with Coupled Epitopes: Testing an Effect on the Immune System

Once protein comprising crossbeta structure is selected for use in immunogenic compositions, based on its properties to efficiently immune potentiate APCs, epitopes are coupled using for example one or more of the aforementioned methods. The immunogenic composition obtained in this way is analyzed for its ability to effectively potentiate the immune system and induce an efficient immune response. For this purpose, example series of experiments are outlined.

Similar to the experimental aims as outlined above in section IV. Determining dimensions of proteins comprising crossbeta structure (molecular/particle size, form) suitable for binding, internalization/engulfment by antigen presenting cells, like dendritic cells, or by accessory innate immune cells, for protein comprising crossbeta structure, now the potency for efficient uptake and processing by APC is tested with the protein comprising crossbeta structure and coupled epitopes (See FIG. 4 for a cartoon). Additional tests with the protein comprising crossbeta structure and coupled epitopes are outlined below.

### Activation of T-Cells by Proteins Comprising Crossbeta Structure and Coupled Epitopes

Analysis of (Primary) T Cell Responses by Immunogenic Compositions Comprising Amino-Acid Sequences with Crossbeta Conformation and Epitopes Coupled to Protein Comprising Crossbeta Structure.

### Isolation and Culture of T Cell Populations.

The ability of immunogenic compositions comprising amino-acid sequences with crossbeta conformation, referred to as ‘crossbeta-antigens’, to induce (primary) T cell responses in vivo is preferably tested in vitro using T cells isolated from immunized animals, for example mammals, for example mice or humans. For example, T cells are isolated from mice or from a human individual. Alternatively, activation of naïve T cells is analyzed upon isolation of T-cells from non-immunized animals, for example mammals, for example from mice or human individuals.

Several methods for T-cell isolation are known and commonly used in practice by persons skilled in the art. Preferably, T cells are isolated from blood or splenocytes, for example from splenocytes isolated from immunized mammals, for example mice. Mammals, for example mice are immunized with antigen, preferably immunogenic compositions comprising protein comprising crossbeta structure and coupled peptide, polypeptide, protein, glycoprotein, protein-DNA complex, protein-membrane complex and/or lipoprotein comprising at least one T-cell epitope motif, preferably once or twice, and cells are isolated preferably between 3 and 14 days after immunization. Preferably, spleen cell suspensions or peripheral blood mononuclear cells are used. Splenocytes are preferably isolated using cell strainers, preferably with a pore size of 100 μm. Preferably, erythrocytes are removed from the cell suspension, preferably by a centrifugation step using Ficoll, or by hemolysis, preferably with a hypotonic buffer, preferably composed of ammonium chloride, preferably at 0.15 mM, and potassium bicarbonate, preferably at 0.1 mM, and ethylendiaminetetaacetic acid, preferably at 0.01 mM.

Subsequently, isolated and washed T-cells are used either directly for analysis of their response towards immunogenic compositions comprising protein comprising crossbeta structure and coupled peptide, polypeptide, protein, glycoprotein, protein-DNA complex, protein-membrane complex and/or lipoprotein comprising at least one T-cell epitope motif, or the isolated and washed T-cells are cultured in appropriate cell culture medium, preferably Dulbecco's Modified Eagle's Medium (DMEM) or RPMI, supplemented with 10% fetal calf serum or human serum, L-glutamine, penicillin, streptomycin and β-mercapto-ethanol, and in appropriate cell culture flasks, for example 96-wells or 24-wells culture systems at appropriate cell density, preferably approximately 5 to 35×10⁶ cells per ml. For example, such analyses are performed in an indirect way with antigen presenting cells included in the analysed cell cultures, and/or directly by assessing responsiveness towards T-cell epitope motifs, for example using peptides of such motifs.

### Analysis of T Cell Response

The number of antigen specific T cells is preferably measured directly, preferably using staining with pre-labeled tetrameric or pentameric MHC molecules, loaded with peptide epitopes derived from the antigen, i.e. T-cell epitope motifs, using a FACS apparatus. Preferably, between 5×10⁵ and 5×10⁶ cells are measured. In addition, the following T cell responses are preferably measured: cytokine production, T cell proliferation and cytotoxic activity of CD8⁺ T cells. For analysis of cytokines isolated cells are preferably cultured for 16 to 48 hrs in the presence of antigen, for example as an immunogenic compositions comprising protein comprising crossbeta structure and coupled peptide, polypeptide, protein, glycoprotein, protein-DNA complex, protein-membrane complex and/or lipoprotein comprising at least one T-cell epitope motif, when antigen presenting cells are included in the analysed cell cultures, or in the presence of T-cell epitope motifs (=T-cell epitopes), when cultures of T-cells only are assessed. Preferably a concentration series of immunogenic composition comprising protein comprising crossbeta structure and T-cell epitope(s), and/or (a) peptide(s) with (an) amino acid sequence(s) of (a) T-cell epitope(s) is tested, preferably at concentrations between 10 ng to 500 μg/ml. For example, such immunogenic composition is provided in the presence of heat shock proteins, such as hsp90, and/or in the presence of a selection of human antibodies, preferably a collection of IVIg, preferably a collection of IVIg selected by a method to enrich for antibodies directed towards crossbeta structure comprising molecules. Induction of cytokine production is preferably measured using a capture method, i.e. using bi-specific antibodies that bind to a common surface molecule on T-cells and to the cytokine to be analyzed on a FACS apparatus. Preferably interferon-γ (IFN-γ), IL-4 and IL-5 are measured and preferably T-cells are co-stained with antibodies for CD4⁺ and CD8⁺, respectively in order to distinguish the phenotype of the responding T cells. Alternatively, cytokine production is for example measured using ELISPOT analysis or ELISA. T cell proliferation is measured for example using ³H-Thymidine incorporation. Preferably proliferation is analyzed after 5-6 days of culture in the presence of antigen, for example provided as the aforementioned immunogenic compositions, when antigen presenting cells are included in the analysed cell cultures, or in the presence of T-cell epitopes, when cultures of T-cells only are assessed, referred to jointly as ‘antigen’ for the two combined possibilities. Preferably a concentration series of such antigen is tested, preferably at concentrations between 10 ng to 500 μg/ml. Preferably the cells are pulsed with, preferably 0.5 μCi/50 μl ³H-Thymidine for the final 6 to 24 hours. Alternatively, proliferation is measured using BrdU or CSFE. For measurement of cytotoxic activity splenocytes isolated from syngeneic animals are for example used as target cells. Target cells are preferably prepared using antigen, for example immunogenic compositions comprising protein comprising crossbeta structure and coupled peptide, polypeptide, protein, glycoprotein, protein-DNA complex, protein-membrane complex and/or lipoprotein comprising at least one T-cell epitope motif, when antigen presenting cells are included in the analysed cell cultures, or using peptides of T-cell epitopes, for 16-48 hr or 1-4 hours, respectively, and loaded with ⁵¹Cr. Preferably a concentration series of such antigen is tested, preferably at concentrations between 10 ng to 500 μg/ml. After removal of free ⁵¹Cr by washing preferably around 3000 cells are used in a 96 well cluster. Lysis of target cells is measured by the release ⁵¹Cr of following the addition of responder cells, derived from the splenocytes stimulated with antigen, for example immunogenic compositions comprising protein comprising crossbeta structure and coupled peptide, polypeptide, protein, glycoprotein, protein-DNA complex, protein-membrane complex and/or lipoprotein comprising at least one T-cell epitope, or with peptides of T-cell epitopes. Preferably a titration of responder cells is tested in ratios of preferably 1:1 to 1:40 with target cells. Alternatively, other target cells, such as tumor cells are for example used, for example E.G7-OVA cells or tumor cells, such as B lymphoma's that can be triggered to present peptides.

For example, mice are immunized with an immunogenic composition comprising ovalbumin T-cell epitopes. Alternatively, T-cell epitopes of PRRS virus proteins, human factor VIII, E2 derived from classical swine fever virus (CSFV), H5 from influenza virus H5N1 strain A/VN/1203/04 or strain A/HK/156/97, or another protein is used in immunogenic compositions comprising protein comprising crossbeta structure and T-cell epitopes, for example. Epitopes coupled to a protein comprising crossbeta structure is the source of T-cell epitopes, and in addition the protein comprising crossbeta structure in some examples comprise T-cell epitopes. Preferably, the antigen protein comprising crossbeta structure and comprising T-cell epitopes, and the coupled peptide(s) are known to be able to generate a T cell response, and/or are predicted to be able to generate a T cell response, preferably by using algorithms and computer based analysis, for example using software such as BIMAS, SYFPEITHI or RANKPEP. For example, such T-cell epitope spanning peptides are derived from pathogens, for example from the proteins of influenza virus, for example from H5N1, for example from the nucleoprotein or for example from proteins of human immunodeficiency virus (HIV), plasmodium falciparum, mycobacterium tuberculosis, PRRS virus protein antigens. Such examples include, but are by no means restricted to, peptide NH₂-AMQMLKETI-COOH [seq. id 5] of the gag24 protein of HIV, and peptides NH₂-IYSTVASSL-COOH [seq. id 6], NH₂-LYQNPTTYI-COOH [seq. id 7], NH₂-TYISVGTST-COOH [seq. id 8], NH₂-KYVKSNRLV-COOH [seq. id 9], NH₂-DYEELKHLL-COOH [seq. id 10], NH₂-SYNNTNQEDL-COOH [seq. id 11], NH₂-TYISVGTSTL-COOH [seq. id 12], and NH₂-KYVKSNRLVL-COOH [seq. id 13] of influenza virus, and in general any known or predicted T-cell epitope spanning peptide is used and coupled to a protein comprising crossbeta structure. Alternatively, such peptides spanning T-cell epitopes are derived from antigens known or predicted to be targets in immunotherapy for cancer or other (human) disease, such as atherosclerosis.

Alternative to primed T cells isolated from immunized non-human animals, or humans which had previously been exposed to an antigen of interest, T cells derived from transgenic animals or T cell clones are for example used. For example, OT-I, OT-II, RF33 or D011.10 cells are used, T cells that are specific for peptides derived from ovalbumin presented in the context of specific MHC class I or MHC class II molecules, respectively peptide NH₂-SIINFEKL-COOH [seq. id 14] (amino acid residues 257-264) and MHC class I allele Kb for RF33, peptide NH₂-VAAHAEINEA-COOH [seq. id 15] (residues 327-337) and MHC class II allele IAd for D011.10, peptide NH₂-SIINFEKL-COOH [seq. id 16] (amino acid residues 257-264) and MHC class I allele Kb for OT-I, peptide NH₂-AAHAEINEAG-COOH [seq. id 17] (residues 328-338) and MHCII allele IAb for OT-II. Alternatively, one of the T cell hybridoma's B3Z, B) 97.10 or 54.8 is for example used. Alternative to splenocytes or monocytes as source of antigen presenting cells, cell lines are for example used as antigen presenting cells, such as for example D1 or DC2.4.

Alternative to in vivo primed T cells, naive T cells are for example used in cultures comprising antigen presenting cells and/or in cultures with T-cell only, to analyse the ability of immunogenic compositions comprising protein comprising crossbeta structure and (coupled) T-cell epitopes, or of peptides spanning T-cell epitopes, to activate the T-cells, respectively. Since the number of T cells specific for the peptides spanning T-cell epitopes is low, the isolated cells are preferably cultured in the presence of mature antigen presenting cells and immunogenic compositions comprising protein comprising crossbeta structure and (coupled) T-cell epitopes for preferably around 1 week and subsequently for a prolonged period, preferably several weeks and preferably in the presence of several cytokines, preferably IL-2, PGE2, TNFα and IL-6 to induce optimal expansion of antigen specific T cells. After expansion, T cells are triggered with peptides spanning T-cell epitopes for preferably 1 to 6 days and analyzed, preferably as described above for primed T cells, for the production of cytokines and/or for their ability to proliferate in response to specific peptides spanning T-cell epitopes.

### MHCI-II (Cross) Presentation of for Example OVA Epitope

Ag processing and presentation in the context of MHCI and MHCII is assayed in vitro by pulsing murine bone marrow derived dendritic cells with ovalbumin and subsequent co-cultured with T cells. Therefore, DC7 cells are washed twice with RPMI⁺ medium supplemented with GMCSF and seeded in 96 well round bottom plates at a concentration of 0.5×10E6 cells/ml or 1×10E6 cells/ml. DCs are pulsed with OVA comprising various crossbeta structures and coupled B-cell epitopes and/or coupled T-cell epitopes at a concentration of 0.1-1-10-100 μg/ml in a total volume of 200 μl RPMI+GMCSF. Excess OVA (400 μg/ml), and peptide T-cell epitope NH₂-SIINFEKLL-COOH/OVA 323-339 (124 μg/ml) are used as positive controls. After 24 hours, pulsed DCs are washed twice with RPMI⁺ medium and co-cultured with 1×10⁵ RF33.70, OT-I and OT-II T cells (DCs derived from C57BL/7), or with D011.10 (DCs derived from Balb/C). Supernatants are harvested from T cell lines after 24 hours at 37° C. and stored at −20° C. until further analysis. Proliferation of OT-I and OT-II T cells is assayed after 48 hours and 72 hours incubation at 37° C. by ³-[H]-thymidine incorporation.

### Analysis of Efficacy of Immunogenic Compositions Comprising Protein Comprising Crossbeta Structure and Coupled T-Cell Epitopes In Vivo.

Immunizations using immunogenic compositions comprising protein comprising crossbeta structure and coupled B-cell epitopes and/or T-cell epitopes (jointly referred to as ‘epitopes’) are preferably aimed at inducing protection against a challenge with a pathogen, and/or aimed at for example treating a disease. Preferably, the capacity of protein comprising crossbeta structure to induce an effective immune response is analyzed in vivo. For example, non-human animals are immunized with immunogenic compositions comprising protein comprising crossbeta structure and coupled epitopes to induce protection against a challenge with a pathogen, for example a virus, bacteria or parasite. For example, non-human mammals are immunized with immunogenic compositions comprising protein comprising crossbeta structure and coupled epitopes, comprising for example H5 and/or peptides thereof, and are subsequently challenged with influenza virus. For example, such challenge is with strain A/HK/156/97 or A/VN/1203/04. In another example, pigs are immunized with immunogenic compositions comprising protein comprising crossbeta structure and coupled epitopes, comprising E2 protein and/or peptides thereof, and or another protein derived from the sequences of the genes encoding proteins of Classical Swine Fever Virus, and challenged with Classical Swine Fever Virus, for example of strain Brescia 456610.

PRRSV Challenge Experiments for Testing the Effectiveness of for Example PRRSV Protein Antigens Comprising Crossbeta Structure and with Coupled Epitopes

Typically, a PRRSV challenge model with pigs is designed as follows. Randomly distributed pigs, for example approximately 30 days old, in groups of typically 4-8 pigs/group are immunized for example at day 0 and day 21 with for example 1) placebo (buffer for injection), 2) positive control vaccine, for example a modified live virus (MLV) PRRS vaccine Pyrsvac-183 (Syva labs, Leon, Spain) and/or a killed virus vaccine with adjuvant: Progressis (Merial labs., Lyon, France) and/or another attenuated live virus vaccine (Ingelvac PRRS MLV), 3) GP5-M heterodimer comprising crossbeta structure with coupled B-cell epitopes and/or with coupled T-cell epitopes, 4) GP4 comprising crossbeta structure with coupled B-cell epitopes and/or with coupled T-cell epitopes, 5) albumin comprising crossbeta structure with coupled B-cell epitopes and/or with coupled T-cell epitopes. For example, at day 28 or 35 or 42, pigs are challenged with autologous PRRSV, and/or with heterologous PRRSV, for example intranassaly, for example with 1-3 ml comprising a dose of 10⁵ TCID50/ml PRRSV. Typically, one to three weeks after the injection of the final antigen dose, lymphocytes are isolated from peripheral blood mononuclear cells (PBMCs) and used for lymphocyte proliferation, cytotoxic T lymphocyte (CTL), and/or cytokine detection assays. During the challenge period, clinical signs, including lack of appetite, depression, lethargy, cough, and breath alterations, are examined and rectal temperatures are measured daily post-challenge. Typically, at day −7, 0, 7, 14, 21, 28, 35, 42, 49, 56, including days during the challenge period, blood samples are taken and serum is isolated for serological tests. Presence of PRRSV neutralizing antibodies is assessed in the collected sera.

Effectiveness of immunization with immunogenic compositions comprising protein comprising crossbeta structure and coupled epitopes, for the treatment of a disease, for example cancer, when for example a tumor antigen is incorporated in the immunogenic composition, or for example atherosclerosis, is preferably analyzed in immunized mammals. For example an effective immune response is determined by performing an in vivo tumor experiment. For example this is performed using an immunogenic composition comprising ovalbumin as the protein comprising crossbeta structure comprising epitopes and coupled epitopes and ovalbumin expressing tumor cells, for example E.G7 cells. After immunization with the immunogenic composition as described, after preferably 7 days, animals are injected intradermally in the back with 5×10⁵ E,G7 tumor cells, which were washed preferably in PBS before injection, preferably in a volume of 200 μl. The mice are then examined in time to monitor tumor growth. The tumor growth is preferably estimated by determining the largest and smallest diameters of the tumors and calculating their size. In another example, the mammals are immunized with immunogenic compositions comprising protein comprising crossbeta structure and coupled epitopes comprising amino-acid sequences of human papilomavirus proteins (HPV), preferably from the E6 or E7 protein, and challenged with HPV. In another example, the mammals, preferably mammals suffering from atherosclerosis, preferably mice or human, are immunized with immunogenic compositions comprising protein comprising crossbeta structure and coupled epitopes, for example in which the protein comprising crossbeta structure is oxidized LDL and/or glycated protein, for example glycated albumin, and analyzed for progression of diseases, preferably by measuring the size of the atherosclerotic plaque, by determining cytokine levels and/or by scoring survival rates.

### A Surrogate Marker for T-Cell Activation in Mice In Vivo: Determination of IgG1/IgG2a Titer Ratio

As a surrogate marker for the occurrence of a humoral response and/or a T-cell activation in vivo upon subjecting an animal, for example a mouse, to immunizations with an immunogenic composition comprising protein comprising crossbeta structure and coupled epitopes, titers of IgG1 and IgG2a are preferably determined using an ELISA with immobilized antigen and dilution series of immune serum, according to methods and protocols known to a person skilled in the art. Increase in IgG1 titers, when compared to pre-immune serum and/or serum of the animal(s) that received placebo, is an indicative measure for the occurrence of a T-helper 2 mediated humoral response, with activation of CD4+ T-helper cells. Increase in IgG2a titers, when compared to pre-immune serum and/or serum of the animal(s) that received placebo, is an indicative measure for the occurrence of a T-helper 1 mediated cellular immune response, with activation of CD8+ cytotoxic T-cells. In addition, total IgG titers are determined as a indicative measure for activation of CD4+ positive T-helper cells.

### T-Cell Activation: Summary

Testing of the immune stimulating efficacy and induction of an effective immune response by immunogenic compositions comprising protein comprising crossbeta structure and coupled B-cell epitopes and/or T-cell epitopes, as outlined in the Examples above comprises two main approaches resulting in the ability of selecting from a plurality of immunogenic compositions those immunogenic compositions having a greater chance of being capable of eliciting and/or stimulating a protective prophylactic immune response and/or a therapeutic immune response in vivo, as compared to the other immunogenic compositions of a plurality of immunogenic compositions. The elicited immune response comprises for example activation of T-cells, for example resulting in a CD4+ T-help response, and/or resulting in a CD8+ cytotoxic T-lymphocyte response. When T-cell epitopes are not known for an antigen and/or when T-cell epitopes are not adequately or not at all predicted by algorithms and computer based analysis, approach I is preferred:

Approach I. Design of Immunogenic Compositions Comprising Protein Comprising Crossbeta Structure and Coupled Epitopes, Checked for Functionality with Cell Cultures of APCS+Naïve and/or Primed T-Cells.

When applying approach I., one predicted and/or putative T-cell epitope and/or series of predicted and/or putative epitopes are incorporated in immunogenic compositions comprising protein comprising crossbeta structure and coupled B-cell epitopes and/or T-cell epitopes. Putative T-cell epitopes are for example obtained by synthesizing peptides covering overlapping sequences of the antigen, comprising preferably the number of amino-acid residues known to be required for presentation by major histocompatibility complexes, for example 5-30 amino-acid residues. The sequence overlap between two adjacent peptides is for example 1-10 amino-acid residues at the N-terminal site of the peptides and/or at the C-terminal site of the peptides.

When T-cell epitopes are known and/or when algorithms and computer based analysis predict T-cell epitopes accurately to a large extent, approach II is preferred:

### Approach II. Design of Ready-to-Use Immunogenic Compositions Comprising Protein Comprising Crossbeta Structure and Coupled Epitopes.

### Peptides Spanning T-Cell Epitopes are

1. predicted T-cell epitopes (MHC class I restricted or MHC class II restricted) obtained using prediction programs, and/or are
2. known T-cell epitopes, like for example, but not limited to, those identified for PRRS virus, H5 or OVA.
   The Known and/or Predicted T-Cell Epitopes are
1. part of the protein comprising crossbeta structure comprising epitopes, and/or are
2. part of coupled epitopes,
   for use as an immunogenic composition in vivo, as a vaccine candidate preceding a challenge with tumor cells or pathogen, and/or with the purpose to obtain primed T-cells, and/or for use as an immunogenic composition in vitro for assessing T-cell activation in vitro, by using co-cultures of APCs and naïve and/or primed T-cells, and/or T-cell clones specific for a known T-cell epitope motif, and/or

a. used as sole peptides
i. having conformations covering those folds that are present when the peptides are presented by major histocompatibility complexes at APCs,
   for assessing direct stimulation of cultured naïve and/or primed T-cells, and/or T-cell clones specific for a known T-cell epitope, in the presence of the selected major histocompatibility complexes, or
ii. comprising crossbeta conformation for 4-75%, and/or
iii. coupled to protein comprising crossbeta structure and comprising epitopes, and/or
iv. coupled to and/or mixed with protein comprising crossbeta structure with unrelated amino-acid sequence with respect to the amino-acid sequence of the parent antigen from which the epitopes are derived,
   for assessing T-cell activation in vivo upon immunization, and/or for obtaining primed T-cells upon immunizations, and/or for assessing T-cell activation in vitro, by using co-cultures of APCs and naïve and/or primed T-cells and/or T-cell clones specific for a known T-cell epitope motif.

Animal or human individuals that have T-cell clones specific for T-cell epitopes under investigation, upon previous immunization with an antigen comprising T-cell epitopes, for example an immunogenic compositions comprising protein comprising crossbeta structure and coupled epitopes, for example upon vaccination and/or for example upon suffering and subsequent recovering from an infection, are serving as a source of T-cells used for the aforementioned experiments comprising cultured primed T-cells.

T-Cell Receptor (TCR) Mimicry by Antibodies as a Tool for Selecting Immunogenic Compositions that Efficiently Stimulate APCs to Process and Present Epitopes

A person skilled in the art can select and/or produce antibodies, which mimic T cell receptors (TCRs) in their binding to antigen fragments presented by APCs in the context of MHC receptors. This means the antibodies recognize antigens that are processed and presented by MHC receptors on antigen presenting cells like dendritic cells. With this type of antibodies the efficiency in which antigen presenting cells present various epitopes, can be checked using antibodies instead of T-cells. For example, T-cell receptor mimicking antibodies are described against the MHC-1 molecule H2-Dd complexed with a peptide derived from the HIV envelope (P18-110). To generate these antibodies mice were immunized with this peptide/MHC-1 complex. Two monoclonal antibodies were selected which were specific for the peptide/MHC-1 complex, namely KP14/1 and KP15/11. A second example of TCR mimicking antibodies is an antibody binding to peptide-HLA-A2 epitopes on dendritic cells (HLA-A2 is a MHC-1 molecule). The vaccine used was a cancer vaccine made with the hCGβ antigen, an antigen expressed by different types of tumors. Two peptide epitopes were recognized, namely a peptide TMT(40-48) (peptide sequence: NH₂-TMTRVLQGV-COOH [seq. Id 18]) and GVL(47-55) (peptide sequence: NH₂-GVLPALPQV-COOH [seq. id 19]). From the hybridoma screen, 15 antibodies were selected which were positive for the TMT-HLA-A2 epitope and 28 which were positive for the GVL-HLA-A2 epitope. A third example is the selection of many MHC-peptide specific antibodies binding to for example three different peptides for gp100, or two for telomerase or peptides from MUC1, HTLV-1, EBV, Influenza or HIV. A fourth example of T-cell receptor mimicking antibodies are antibodies specific for a novel tumor antigen, named TCRγ alternative reading frame protein (TARP), which is expressed on prostate and breast cancer cells. Antibodies against HLA-A2/peptide epitopes were selected. A fifth example is the crystal structure determination of a TCR-like antibody Fab fragment bound to an ovalbumin peptide in complex with the H-2 Kb MHC-1 molecule. The selected specific antibody was 25-D1.16. The ovalbumin peptide used was the pOV8 peptide (NH₂-SIINFEKL-COOH). The antibody was generated by immunizing mice with whole antigen presenting cells bearing the pOV8 complexes.

Typically, TCR mimicking antibodies are used in the examples outlined above, that are specific for the epitopes coupled to the protein comprising crossbeta structure. For example, TCR mimicking antibodies binding to epitopes as outlined in Table 1 and 2 and in the text of the specification and examples.

Testing for Humoral Responses in Animals after Immunizations with Immunogenic Compositions Comprising Protein Comprising Crossbeta Structure and Coupled Epitopes

Upon immunization of animals, like for example mice, rat, rabbits, pigs, cows, or humans, with immunogenic compositions comprising protein comprising crossbeta structure and coupled epitopes, induction of a humoral response is assessed by determining antibody titers, for example IgG titers, IgM titers, total Ig titers, and/or by determining titers of functional antibodies. For example, virus neutralizing antibody titers are determined in serum or blood of animals immunized with immunogenic compositions comprising virus epitopes. For example, bactericidal antibody titers are determined in serum or blood of animals immunized with immunogenic compositions comprising virus epitopes. For example, rabbits are immunized with protein comprising crossbeta structure with coupled PRRSV B-cell epitopes, and antibody titers against the epitopes is analyzed in immune serum.

### BRIEF DESCRIPTION OF THE DRAWINGS

FIG. 1. Crossbeta Epitopes.

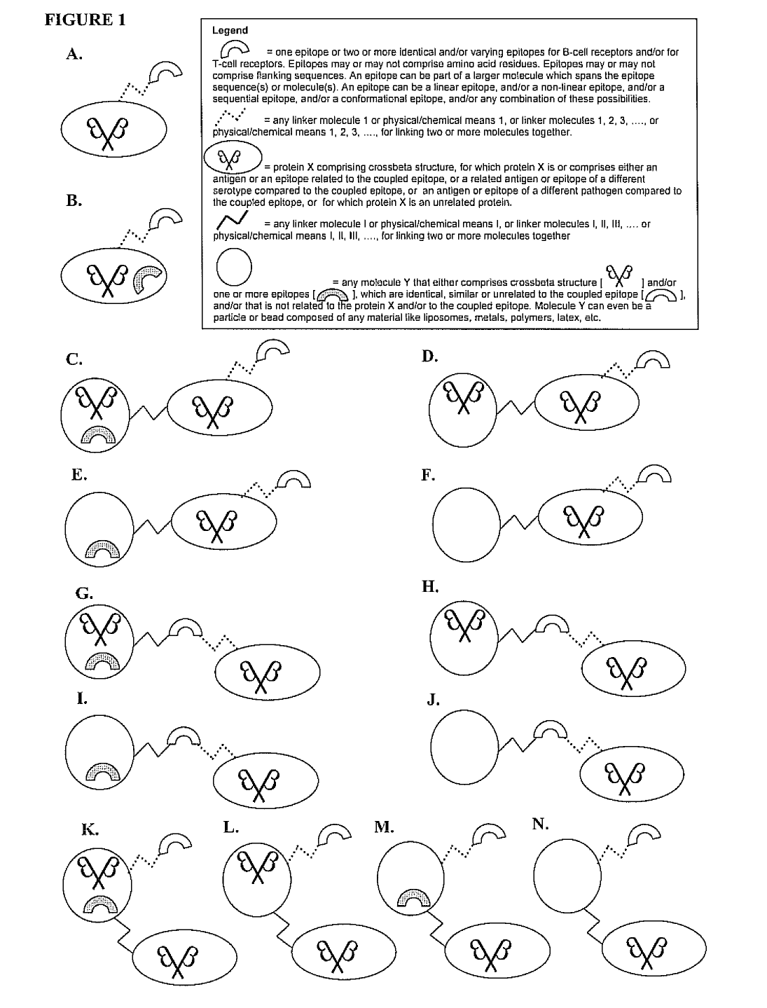

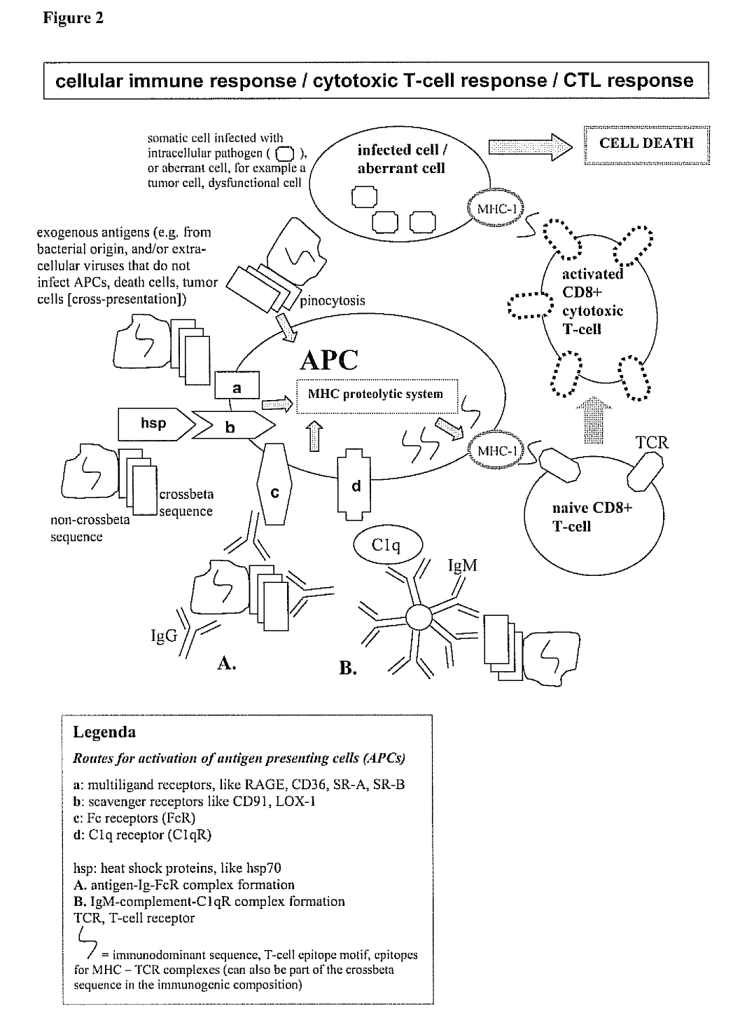

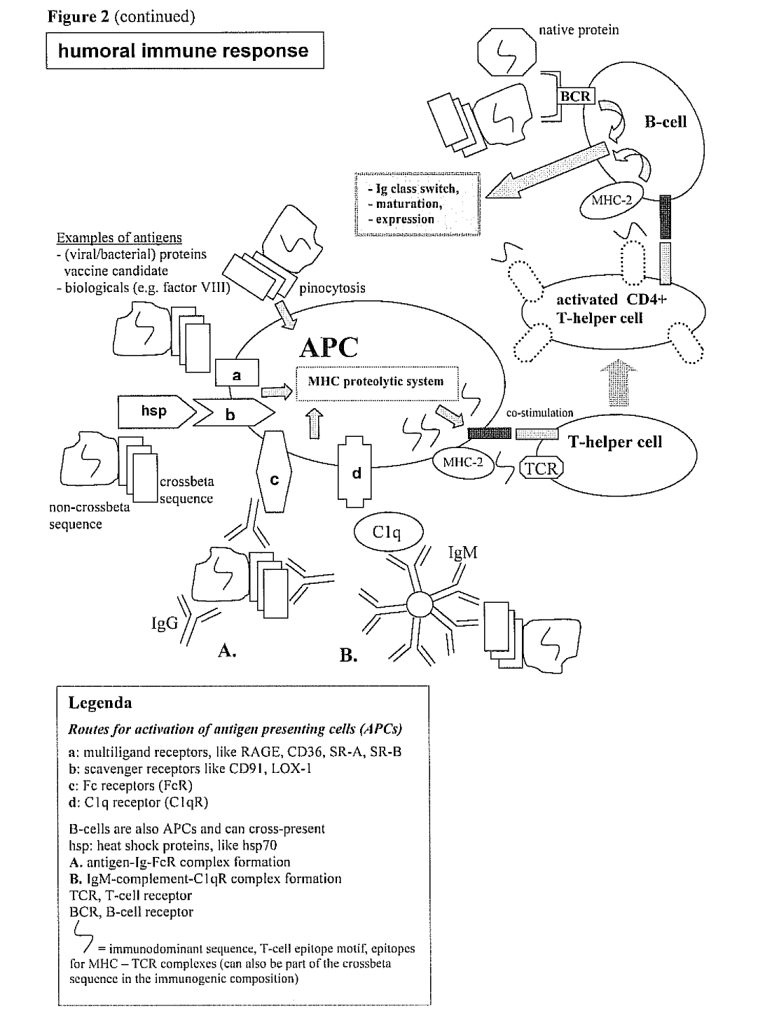

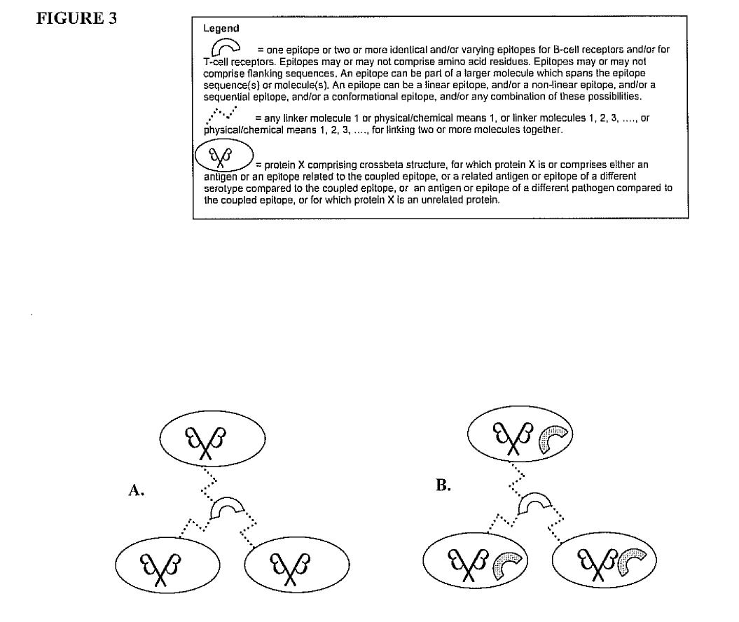

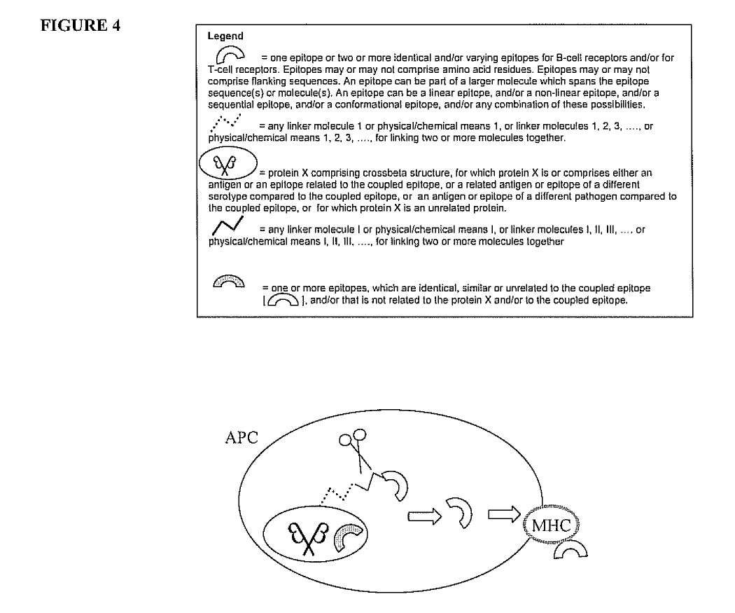

Immunogenic compositions comprise protein X with crossbeta structure linked or coupled to molecules comprising one or more epitopes for one or more different B-cell receptors and/or one or more epitopes for one or more different T-cell receptors that are provided in a series of varying appearances. The legend explains the non-limiting series of examples of possible varying appearances that are depicted in FIG. 1.A-M. Of course, for example with Crossbeta Epitope (CE) variant A., depicted in FIG. 1A., also more than one epitope can be coupled to the protein comprising crossbeta upon linking more molecules comprising the epitope to various coupling sites of the protein comprising crossbeta. Furthermore, when more than one epitope is coupled through more than one linker molecule, these more than one epitope molecules can be identical or similar or unrelated to each other with respect to sequence and/or type of molecule. Linker molecules may comprise scissile bonds providing the ability to release epitopes. Linker molecules may be supporting structures capable of presenting a conformational and/or discontinuous epitope.

FIG. 2. Schematic overview of a cellular immune response and a humoral immune response.

FIG. 3. Epitope with one or several coupled protein molecules comprising crossbeta structure. Drawing depicting coupling of an epitope simultaneously to one or more protein molecules comprising crossbeta structure, either or not through a linker molecule. The carrier protein comprising crossbeta structure may (A.) or may not (B.) comprise an epitope, to which (partly) the immune response is mounted.

FIG. 4. Processing of the epitope coupled to a protein comprising crossbeta structure, after uptake by an antigen presenting cell. The scissors cartoon indicates release of the epitope from the protein comprising crossbeta structure.

TABLE 1
B-cell epitopes

| | | | | Known | Functional |
|---|---|---|---|---|---|
| disease | pathogen | protein | Epitope sequence | antibody | antibody |
| HIV | HIV-I | Gp41 | Minimum epitope sequence | | 2F5 |
| infection | | | within residues 660-680 in | | |
| | | | the membrane proximal region | | |
| | | | of gp41 of the potent HIV | | |
| | | | neutralizing antibody 2F5, | | |
| HIV | HIV-I | Gp41 | Minimum epitope sequence | | 4E10 |
| infection | | | within residues 660-680 in | | |
| | | | the membrane proximal region | | |
| | | | of gp41 of the potent HIV | | |
| | | | neutralizing antibody 4E10. | | |
| HIV | HIV-I | Gp41 | ERDRD | | ERDRD elicits |
| infection | | | Decoy epitope IEEE | | neutralizing Abs |
| PRRS | PRRSV | ORF 2, 3, | Table 1 Oleksiewicz et al | | |
| | | 4, 5 and 6 | 2002 and FIG. 2 Oleksiewicz | | |
| | | | et al 2001 | | |
| PRRS | PRRSV | Nucleocapsid | IQTAFNQGA | | |
| | | protein | (79-87 of CH-1a N protein | | |
| | | (N, ORF6) | | | |
| PRRS | PRRSV | GP5 (ORFS) | Neutralizing epitope is | | Gp5 induces |
| | | | close to glycosylation site | | neutralizing Abs. |
| | | | 4 immunodominant peptides | | |
| | | | (poster Vashisht) | | |
| PRRS | PRRSV | GP5 | Decoy epitope close to | | |
| | | | neutralizing epitope | | |
| PRRS | PRRSV | GP5 | R¹⁵¹LYRWR¹⁵⁶ | | |
| | | | K¹⁶⁵VEVEGHLIDLKRVVL¹⁸⁰ | | |
| | | | Q¹⁹⁶wGRL²⁰⁰/Q¹⁹⁶GRp²⁰⁰ | | |
| PRRS | PRRSV | M (ORF7) | B-cell linear epitope | | Neutralzing ab are |
| | | | | | raised against M5 |
| PRRS | PRRSV | GP3 | Two 15 aa peptides | | GP3 induces PRRSV |
| | | | corresponding to two | | neutralizing Abs. |
| | | | immunodominant epitopes | | |
| | | | (De Lima) | | |
| PRRS | PRRSV | GP5 | Epitope A: (A/V²⁷)LVN³⁰ | | B elicits neutralizing |
| | | | Epitope B: S37H(F/L³⁹)-I⁴²YNL⁴⁵ | | Abs, A elicits non |
| | | | | | neutralizing Abs, A is |
| | | | | | highly immunodominant |
| PRRS | PRRSV | GP4 (ORF4) | GP4 has a domain that | | |
| | | | elicits neutralizing Abs. | | |
| PRRS | PRRSV | Nucleocapsid | 79-IQTAFNQGA-87 (strain | | |
| | | (N) protein | CH-1a) (highly conserved | | |
| | | | among North American and | | |
| | | | European isolates) | | |
| PRRS | PRRSV | Gp4 | Neutralizing epitopes | | Neutralizing |
| | | | | | antibodies |
| PRRS | PRRSV | Gp5-M | Epitopes for neutralizing | | Neutralizing |
| | | heterodimer | antibodies | | antibodies |
| PCVAD | PVC2 | Capsid | Aa 47-63 (structural epitope) | | |
| | | protein | Aa 165-200 | | |
| | | | Last 4 aa at the C-terminus | | |
| HPV | HPV16 | E7 | Linear B epitope ⁴⁴QAEPD⁴⁸ | | |
| infection | | | | | |
| HPV | HPV16 | E6 | C-term part of E6; E6:10 | IgA | |
| infection | | | | | |
| HPV | HPV16 | EI | Middle part of EI; EI:19 | IgG | |
| infection | | | | | |
| Cervical | HPV | L2 | QLYKTCKQAGTCPPDIIPKV | | Neutralizing Ab |
| carcinoma | | | | | conserved between |
| | | | | | diverse HPV types |
| Cervical | HPV16 | E7 | E7₄₉₋₅₇ RAHYNIVTF | | H-2b-restricted |
| carcinoma | | | | | |
| Cervical | HPV16 | L1 | L1₁₆₅₋₁₇₃ AGVDNREC | | H-2b-restricted |
| carcinoma | | | | | |
| Cervical | HPV16 | HPV16 | L1 | | virus neutralizing |
| carcinoma | | major | | | antibodies |
| | | capsid | | | |
| | | protein L1 | | | |
| Cervical | HPV16 | E7 | E7 58-68 CCKCDSTLRLC | | HLA-DR17 |
| carcinoma | | | | | |
| Cervical | HPV16 | E6/E7 | (E7:11-20; YMLDLQPETT) | | CTL/Th |
| carcinoma | | | (E7:82-90; LLMGTLGIV) | | HLA-A*0201 |
| | | | (E7:86-93; TLGIVCPI) | | |
| | | | (E6:29-38; TIHDIILECV). | | |
| Cervical | HPV16 | E7 | E7⁴³⁻⁷⁷ 35-residue long peptide | | H-2b-restricted |
| carcinoma | | | GQAEPDRAHYNIVTFCCKCD | | |
| | | | STLRLCVQSTHVDIR, | | |
| TB | M. | Ag85A | LTSELPGWLQANRHVKPTGS | | H-2d-restricted |
| | tuberculosis | | | | |
| TB | M. | Ag85A | Ag85A:1-19 | | H-2b-restricted |
| | tuberculosis | | | | |
| TB | M. | TB10 | TB10:4-12 and 73-87 | | H-2b-restricted |
| | tuberculosis | | | | |
| TB | M. | MPT51 | GGPHAVYLL | | H-2d-restricted |
| | tuberculosis | | | | |
| TB | M. | Ag85B | Ag85B₂₄₀₋₂₅₄ | | I-A^{b}-restricted |
| | tuberculosis | | FQDAYNAAGGHNAVF) | | |

TABLE 2
T-cell epitopes

| | | | | Known | Functional |
|---|---|---|---|---|---|
| disease | pathogen | protein | Epitope sequence | specific T-Cell | antibody |
| PRRS | PRRSV | Nsp2 (non | A cluster of B-cell | Certain deletion | |
| | | structural | epitopes on nsp2. | regions map to | |
| | | proteins) | Especially in | potential T-cell | |
| | | | hypervariability region. | epitopes. | |
| PRRS | PRRSV | Nsp1/nsp7 | | | |
| PRRS | PRRSV | GP5 | P6 peptide | Protective | |
| | | | | immune | |
| | | | | response by pig | |
| | | | | T lymphocytes | |
| HPV | HPV16 | E7 | Th epitope ⁴⁸DRAHYNI⁵⁴ | Th1 + Th2 | |
| infection, | | | CTL epitope ⁴⁹RAHYNIVTF⁵⁷ | | |
| cervical | | | | | |
| cancer | | | | | |
| HPV | HPV16 | E7 | E7 44-52 | CTL response, | |
| infection, | | | | Presented by | |
| cervical | | | | HLA-B18 | |
| cancer | | | | molecule | |
| HPV | HPV16 | E6 | E6 80-88 | CTL response, | |
| infection, | | | | Presented by | |
| cervical | | | | HLA-B18 | |
| cancer | | | | molecule | |
| HPV | HPV16 | | HPV16 E711-20 | | |
| infection, | | | | | |
| cervical | | | | | |
| cancer | | | | | |
| HPV | HPV16 | | HPV16 E786-93 | | |
| infection, | | | | | |
| cervical | | | | | |
| cancer | | | | | |
| HPV | HPV16 | L1 | L1 | L1 specific | |
| infection, | | | | CTLs | |
| cervical | | | | | |
| cancer | | | | | |
| Hepatitis B | HBV | | HBsAg/HBcAg | Viral specific | |
| | WHB | | WHsAg | CD8 T Cell | |
| Genital | HSV-2 | Recombinant | | Viral specific | |
| Herpes | | glycoprotein | | CD8 T Cell | |
| | | D, (rgD2) | | | |
| HIV | HIV-I | Gp120-gp41 | | CDS⁺ | |
| infection | | | | | |
| HIV | HIV-I | Gag | | CDS⁺ | |
| infection | | | | | |
| HIV | HIV-I | Nef/pol | | CDS⁺ | |
| infection | | | | | |
| Flue | Influenza | Nucleoprotein, | Described in Influenze | HLA class I | |
| | | NP | Sequence Database | | |

### REFERENCES

1. www.immuneepitope.org/export/doc/influenza/index.html [website with influenza antibody and T-cell epitopes]
2. Hoffmann et al., PNAS 2005, V102, no. 36, pp. 12915-12920 [article on influenza epitopes]
3. Bui et al., PNAS 2007, V104, no. 1, pp. 246-251 [article on influenza epitopes]
4. Wang et al., Vaccine 2007, V25, pp. 2823-2831 [epitopes in influenza A haemagglutinin for human CTL response]
5. Rapid and Quantitative Cyclization of Multiple Peptide Loop onto Synthetic Scaffolds for Structural Mimicry of Protein Surfaces, Timmerman et al, Chem. Biochem. 2005, 6, 1-5
6. Functional reconstruction and synthetic mimicry of a conformational epitope using CLIPS™ technology, Timmerman et al, J Mol Recognit. 2007, 20, 283-299
7. An et al., Virus Genes 31:1, pp. 81-87, 2005 [PRRSV epitope]
8. Kranenburg et al., Tissue-type plasminogen activator is a multiligand cross-beta structure receptor. Curr. Biol. 2002 Oct. 29; 12(21):1833-9.

1. A method for producing an immunogenic composition, the method comprising:
providing a protein,
inducing a crossbeta structure in said protein and providing said protein with at least one exogenous epitope to form an epitope-protein complex and
combining said epitope-protein complex with a vehicle suitable for administration to a subject.

2. A method according to claim 1, wherein said at least one epitope is brought in an immunogenic form by a supporting structure.

3. The method according to claim 1, wherein said at least one epitope is a discontinuous epitope, the constituting parts of which are brought together on a supporting structure.

4. The method according to claim 1, wherein said protein and said at least one epitope are from the same antigen, pathogen, and/or aberrant cell.

5. The method according to claim 1, wherein said crossbeta structure comprising protein comprises relevant endogenous epitopes.

6. The method according to claim 1, wherein said crossbeta structure comprising protein comprises no relevant endogenous epitopes.

7. The method according to claim 1, wherein said at least one epitope comprises a T-cell epitope.

8. A method according to claim 7, wherein said T-cell epitope comprises anchor residues, cleavage sites and/or processing sites for uptake and presentation by antigen presenting cells.

9. The method according to claim 1, wherein said at least one epitope comprises a B-cell epitope.

10. The method according to claim 1, wherein said protein is provided with several exogenous epitopes.

11. The method according to claim 1, wherein at least one crossbeta structure is induced in said protein before said protein is provided with at least one exogenous epitope.

12. An epitope-protein complex produced by the method according to claim 1.

13. An immunogenic composition consisting of epitope-protein complexes produced by a method according to claim 1 and a vehicle suitable for administration.

14. An immunogenic composition according to claim 13, wherein said vehicle is water for injection.

15. The immunogenic composition according to claim 13, which is a vaccine.

16. A method for producing antibodies against at least one desire epitope, comprising:
preparing an immunogenic composition according to claim 13,
administering said composition to an nonhuman mammal,
isolating B-cells from said nonhuman mammal, and
generating antibody producing cells and/or antibodies from said B cells.

17. The method according to claim 9, wherein the B-cell epitope is provided with B-cell processing sites.

18. The method according to claim 10, wherein the protein is provided with both B-cell and T-cell epitopes.
